(19) United States
(12) Patent Application Publication
(10) Pub. No.: US 2026/0108434 A1
(43) Pub. Date: Apr. 23, 2026

(54) DENTIFRICE COMPRISING CARBOXYLIC ACID OR ALKALI METAL SALT THEREOF AND A SOURCE OF FREE FLUORIDE IONS

(71) Applicant: Haleon UK IP Limited, Weybridge Surrey (GB)

(72) Inventors: Donal GREY, Weybridge Surrey (GB); Rachel TAYLOR, Weybridge Surrey (GB); Debbie HUNT, Weybridge Surrey (GB); Matthew HOOKHAM, Weybridge Surrey (GB); Jonathan Edward CREETH, Surrey (GB); Shazada Yassar KHAN, Surrey (GB); Richard LYNCH, Surrey (GB); David URQUHART, Surrey (GB)

(21) Appl. No.: 19/414,831

(22) Filed: Dec. 10, 2025

Related U.S. Application Data
(63) Continuation-in-part of application No. 17/252,372.

(30) Foreign Application Priority Data
Jul. 5, 2018 (GB) 1811061.9

(51) Int. Cl. A61K 8/21; A61K 8/365; A61K 8/81; A61Q 11/00

(52) U.S. Cl. CPC A61K 8/21; A61K 8/365; A61K 8/8164; A61Q 11/00; A61K 2800/591

(57) ABSTRACT

A dentifrice composition is described comprising a copolymer of MVE with maleic acid; lactic acid or alkali metal salt thereof; and a source of fluoride ions, wherein the copolymer is present in an amount of range 0.9% to 1.1% by weight of the total composition; wherein the lactic acid or alkali metal salt thereof is present in an amount of 1% to 4% by weight of the total composition, wherein the dentifrice composition is mildly acidic, having a slurry pH in the range 5.0 to 6.5. The composition enhances fluoride uptake into teeth and provides protection against acidic challenges.

### CROSS REFERENCE TO RELATED APPLICATIONS

This application is a continuation-in-part of U.S. patent application Ser. No. 17/252,372, filed Dec. 15, 2020, which is a U.S. national phase entry under 35 U.S.C. § 371 of International Application No. PCT/EP2019/067791, filed on Jul. 3, 2019, which claims priority to GB1811061.9, filed on Jul. 5, 2018, each of which is incorporated by reference herein in its entirety.

### FIELD

This disclosure generally relates to a dentifrice composition for strengthening and protecting enamel of natural teeth, thereby providing protection against acidic challenges.

### BACKGROUND

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth decay may occur as a result of dental caries, which is a multifactorial disease where bacterial acids such as lactic acid produced by metabolism of dietary sugars leads to sub surface demineralization that does not fully remineralize in between sugar exposures, resulting in progressive tissue loss and eventually cavity formation. The presence of a plaque biofilm is a prerequisite for dental caries, and acidogenic bacteria such as Streptococcus mutans may become pathogenic when levels of sugars (i.e. easily fermentable carbohydrate such as sucrose), are elevated for extended periods of time.

Even in the absence of a plaque biofilm, loss of dental hard tissues can occur as a result of acid erosion and/or physical tooth wear; these processes are believed to act synergistically. Exposure of the dental hard tissues to acid can cause demineralization, resulting in surface softening and a decrease in mineral density. This softened mineral is vulnerable to wear from physical contact. Under normal physiological conditions, partially demineralized tissues self-repair through the demineralizing effects of saliva. Saliva is supersaturated with respect to calcium and phosphate, and in healthy individuals, saliva secretion serves to wash out the acid challenge, and to raise the pH so as to alter the equilibrium in favor of mineral deposition.

Dental erosion (i.e. acid erosion or acid wear) is a surface phenomenon that involves demineralization, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCl) produced by the stomach, which may enter the oral cavity through an involuntary response such as gastroesophageal reflux, or through an induced response as may be encountered in sufferers of bulimia.

Tooth wear (i.e. physical tooth wear) is caused by attrition and/or abrasion. Attrition occurs when tooth surfaces rub against each other, a form of two-body wear. An often-dramatic example is that observed in subjects with bruxism, a tooth-grinding habit during sleep where the applied forces are high, and is characterized by accelerated wear, particularly on the occlusal surfaces. Abrasion typically occurs as a result of three-body wear, and the most common example is that associated with brushing with a toothpaste. In the case of fully mineralized enamel, levels of wear caused by commercially available toothpastes are minimal and of little or no clinical consequence. However, if enamel has been demineralized and softened by exposure to an erosive challenge, the enamel becomes more susceptible to wear. Enamel is thinnest at its junction with the dentine, which in health is located just below the gum margin. However, gum recession (especially associated with aging) can expose the enamel-dentine junction and wear of enamel in this region can expose dentine, leading to hypersensitivity, as described below.

Dentine is a vital tissue that in vivo is normally covered by enamel or cementum depending on the location, i.e. crown versus root respectively. Dentine has a much higher organic content than enamel and its structure is characterized by the presence of fluid-filled tubules that run from the surface of the dentine-enamel or dentine-cementum junction to the pulp interface. Dentine is much softer than enamel and consequently is more susceptible to wear. Subjects with exposed dentine should avoid the use of highly abrasive toothpastes. Again, softening of dentine by an erosive challenge will increase susceptibility of the tissue to wear. It is widely accepted that the origins of dentine hypersensitivity relate to changes in fluid flow in exposed tubules, (the hydrodynamic theory), that result in stimulation of mechanoreceptors thought to be located close to the pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become completely occluded with mineralized tissue. The formation of reparative dentine in response to trauma or chemical irritation of the pulp is also well-documented. Nonetheless, an erosive challenge can remove the smear layer and tubule “plugs” releasing dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the pulp interface, progressive dentine wear can result in an increase in hypersensitivity, especially in cases where dentine wear is rapid.

Erosion and/or acid-mediated tooth wear are therefore primary etiological factors in the development of dentine hypersensitivity.

It has been claimed that an increased intake of dietary acids and a move away from formalized meal times has been accompanied by a rise in the incidence of dental erosion and tooth wear in the populations of developed countries. In view of this, oral care compositions which can help prevent dental erosion and tooth wear and which provide protection from dental caries would be advantageous.

Oral care compositions often contain a source of fluoride ions for promoting remineralization of teeth and for increasing the acid resistance of dental hard tissues. To be effective the fluoride ions must be available for uptake into the dental hard tissues being treated.

There is a need for a dentifrice that enhances the uptake of fluoride ions while at the same time improves enamel solubility reduction without adverse impacts on fluoride uptake.

### SUMMARY

In one aspect the present disclosure is based on the discovery that incorporation of a particular carboxylic acid(s) as described herein in a mildly acidic dentifrice composition comprising a source of fluoride ions, advantageously enhances the uptake of fluoride ions into dental enamel when compared to the same composition under a neutral pH, or when compared to the same mildly acidic composition but containing a different carboxylic acid (such as malic acid), or an inorganic acid (such as phosphoric acid).

In a further aspect the present disclosure is based on the discovery that incorporation of a copolymer of methyl vinyl ether with maleic anhydride or acid provides a further benefit of significantly increasing enamel solubility reduction without adversely impacting on uptake of fluoride.

In another aspect disclosed herein is a dentifrice composition comprising a carboxylic acid or alkali metal salt thereof wherein the acid is selected from the group consisting of malonic acid, glutaric acid, tartaric acid, lactic acid and mixtures thereof; and a source of free fluoride ions; and wherein the composition has a slurry pH in the range from greater than 5.0 to less than 6.5.

In a further aspect disclosed herein is a dentifrice composition comprising a carboxylic acid or alkali metal salt thereof wherein the acid is selected from the group consisting of malonic acid, glutaric acid, tartaric acid, lactic acid and mixtures thereof; a source of free fluoride ions; and a copolymer of methyl vinyl ether with maleic anhydride or acid; and wherein the composition has a slurry pH in the range from greater than 5.0 to less than to 6.5.

In yet another aspect, disclosed herein is a dentifrice composition comprising: a copolymer of MVE with maleic acid; lactic acid or alkali metal salt thereof; and a source of fluoride ions; wherein the copolymer is present in an amount of range 0.9% to 1.1% by weight of the total composition; wherein the lactic acid or alkali metal salt thereof is present in an amount of 1% to 4% by weight of the total composition; and wherein the composition has a slurry pH in the range from 5.0 to 6.5.

In one embodiment, the copolymer is present in an amount of 0.95% to 1.05% by weight of the total composition. In one embodiment, the lactic acid or alkali metal salt thereof is present in an amount of 2% to 3% by weight of the composition. In one embodiment, the alkali metal salt is sodium lactate present in an amount of 2.5% by weight of the total composition. In another embodiment, the source of free fluoride ions is an alkali metal fluoride. In yet another embodiment, the alkali metal fluoride is sodium fluoride present in an amount of 0.05% to 0.5% by weight of the composition.

In one embodiment, the composition has a slurry pH in the range from 5.4 to 6.3. In another embodiment, the composition comprises a pH modifying agent. In another embodiment, the pH modifying agent is sodium hydroxide.

In one embodiment, the copolymer of MVE with maleic acid is a 1:1 copolymer. In another embodiment, copolymer has a molecular weight in the range 100,000 to 2,000,000.

In another embodiment, the composition further comprises a desensitizing agent.

In one embodiment, the surface microhardness reduction of enamel treated with the composition following a 5 day pH cycling treatment with 1% citric acid solution at a pH of 3.9 is less than 55%. In yet another embodiment, the enamel fluoride concentration of enamel treated with the composition following 5 days of pH cycling treatment with 1% citric acid solution at a pH of 3.9 is at least 175 ppm.

Such compositions are of use in protecting teeth against dental erosion. Such compositions are also of use in protecting teeth against dental caries. In one aspect, disclosed herein is a method of protecting teeth against dental erosion, comprising administering a dentifrice composition disclosed herein. In another aspect, disclosed herein is a method of protecting teeth against dental caries, comprising administering a dentifrice composition disclosed herein.

### BRIEF DESCRIPTION OF THE DRAWINGS

FIG. 1 is a graphical representation of the effect of malonic acid and pH on Enamel Fluoride Uptake (EFU) (S=slurry, values are mean, ±s.e.).

FIG. 2 is a graphical representation of the effect of malonic acid and citric acid (at pH 5.50) on EFU (S=slurry, values are mean, ±s.e.).

FIG. 3 is a graphical representation of the effect of malonic acid and pH on EFU (mean, ±s.e.).

FIG. 4 is a graphical representation of the effect of carboxylic acids and phosphoric acid on EFU (F=formulation).

FIG. 5 is a graphical representation of the effect of lactic acid and pH on EFU (F=formulation; mean, ±s.e.).

FIG. 6 is a graphical representation of the effect of PVM/MA (polyvinyl methyl ether/maleic acid) (pH 6.2) on EFU (F=formulation; mean, ±s.e.).

FIG. 7 is a graphical representation of the Effect of PVM/MA (pH 6.2) on Enamel Solubility Reduction (ESR) (F=formulation; mean, ±s.e.).

FIG. 8 is a graphical representation of Surface Microhardness Recovery (SMHR) after 4 hrs Remineralization (ITT Population, N=62).

FIG. 9 is a graphical representation of mean percent relative erosion resistance (% RER) after 4 hrs Remineralization (ITT Population, N=62).

FIG. 10 is a graphical representation of EFU after 4 hrs Remineralization (ITT Population, N=62).

FIG. 11 is a graphical representation of tissue loss after treatment of human enamel with dentifrices followed by an erosive challenge.

FIG. 12 is a graphical representation of the variation of mean fluoride uptake over 50 μm depth.

FIG. 13 is a graphical representation of the mean relative ⁴⁴Ca uptake over 20 μm depth.

FIG. 14 is a graphical representation of Surface Microhardness Reduction of Enamel following 5-day pH cycling treatment regimen.

FIG. 15 is a graphical representation of Sound Enamel Hardness (SEH) retention.

FIG. 16 is a graphical representation of enamel fluoride concentration (in ppm F) following 5-day pH cycling treatment regimen.

### DETAILED DESCRIPTION

Embodiments described herein can be understood more readily by reference to the following detailed description and examples and their previous and following descriptions. Elements and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

When a range of integers is given, the range includes any number falling within the range and the numbers defining ends of the range. For example, when the terms “integer from 1 to 20” is used, the integers included in the range are 1, 2, 3, 4, 5, 6, 7, 8, 9, 10, etc., up to and including 20. All ranges disclosed herein are also to be considered to include the end points of the range, unless expressly stated otherwise. For example, a range of “between 5 and 10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

Furthermore, the terms “substantially,” “approximately,” and “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount. The term “consists essentially of” (and grammatical variants) shall be given its ordinary meaning and shall also mean that the composition or method referred to can contain additional components as long as the additional components do not materially alter the composition or method. The term “consists of” (and grammatical variants) shall be given its ordinary meaning and shall also mean that the composition or method referred to is closed to additional components. The term “comprising” (and grammatical variants) shall be given its ordinary meaning and shall also mean that the composition or method referred to is open to contain additional components.

It is also to be understood that the article “a” or “an” refers to “at least one,” unless the context of a particular use requires otherwise.

Also as used herein, “and/or” refers broadly to and encompasses any and all possible combinations of one or more of the associated listed items, as well as the lack of combinations when interpreted in the alternative (“or”).

### Definitions

The term “slurry pH” as used herein, refers to the pH of a slurry comprising a dentifrice composition disclosed herein slurried with distilled water in a 1:3 weight ratio (composition:distilled water). The pH is determined using a standard pH meter.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like this application controls. Unless otherwise defined, all technical and scientific terms used herein have the same meaning as commonly understood by one of ordinary skill in this field. All publications, patent applications, patents, and other references mentioned herein are incorporated by reference in their entirety.

In one aspect, disclosed herein is a dentifrice composition comprising a carboxylic acid or alkali metal salt thereof wherein the acid is selected from the list consisting of malonic acid, glutaric acid, tartaric acid, lactic acid and mixtures thereof; and a source of free fluoride ions, and wherein the composition has a slurry pH in the range from greater than 5.0 to less than 6.5.

In one embodiment, the composition comprises a carboxylic acid or alkali metal salt thereof wherein the acid is selected from the group consisting of malonic acid, glutaric acid, tartaric acid, lactic acid and mixtures thereof. In one embodiment the carboxylic acid is lactic acid or an alkali metal salt thereof. Typical examples of suitable alkali metal salts include the sodium and potassium salts of the said carboxylic acids. In one embodiment the alkali metal salt is the potassium salt(s) of malonic, glutaric, tartaric, lactic acids and mixtures thereof. In one embodiment the alkali metal salt is selected from the sodium salt(s) of malonic, glutaric, tartaric, lactic acids and mixtures thereof. In one embodiment the carboxylic acid salt is potassium lactate. In one embodiment the carboxylic acid salt is sodium lactate. The carboxylic acid or salt may be provided in the form of a solid or an aqueous solution, e.g. sodium lactate solution (60% w/w).

In some embodiments, the carboxylic acid or alkali metal salt thereof is present in an amount of 0.5% to 5.0% by weight of the total composition, for example 1.0% to 4.5% or 1.5% to 3.0% by weight of the total composition. In one embodiment, the amount of carboxylic acid or alkali metal salt is 2.0% by weight of the acid or 2.5% by weight of the salt. In one embodiment, the composition comprises lactic acid or an alkali metal salt thereof in an amount of 1% to 4% or 2% to 3% by weight of the total composition. In another embodiment the alkali metal salt is sodium lactate, which is present in the amount of 2.5% by weight of the total composition.

In one embodiment, the composition comprises a source of free fluoride ions. Suitable examples of a source of free fluoride ions include an alkali metal fluoride such as sodium or potassium fluoride, polyvalent metal ion fluoride salts such as stannous fluoride, or salts of fluoride with cationic organic ions such as ammonium fluoride or bis-(hydroxyethyl) amino-propyl-N-hydroxyethyloctadecylamine-dihydrofluoride, (amine fluoride) or a mixture thereof in an amount to provide from 25 to 5000 ppm of fluoride ions or from 100 to 1500 ppm. In one embodiment the source of free fluoride ions is stannous fluoride. In one embodiment the source of free fluoride ions is not stannous fluoride. In one embodiment the source of free fluoride ions is an alkali metal fluoride such as sodium fluoride. In some embodiments, the composition contains from 0.05% to 0.5% by weight of sodium fluoride, e.g. 0.1% by weight (equating to 450 ppm of fluoride ions), 0.205% by weight (equating to 927 ppm of fluoride ions), 0.2542% by weight (equating to 1150 ppm of fluoride ions) or 0.3152% by weight (equating to 1426 ppm of fluoride ions). In some embodiments, alkali metal fluoride, such as sodium fluoride, is present in an amount of 0.2% to 0.3% by weight of the composition.

In yet another embodiment, the composition is mildly acidic and has a slurry pH in the range from greater than 5.0 to less than 6.5. In one embodiment, the composition has a pH from pH 5.1 to 6.4, 5.4 to 6.3, 5.5 to 6.2 or 6.1 to 6.3. In one embodiment, the pH is about 6.2.

In one embodiment, the dentifrice composition comprises a pH modifying agent. In some embodiments, the pH modifying agent is used to adjust the pH of the composition to the desired pH. Such pH modifying agents can include, but are not limited to, alkali metal hydroxides, such as for example, sodium hydroxide and potassium hydroxide, or inorganic acids, such as for example, hydrochloric acid or sulfuric acid. In one embodiment the pH modifying agent is sodium hydroxide. In some embodiments, the pH modifying agent is present in the dentifrice composition in an amount from 0.005% to 5% by weight of the composition, from 0.01% to 2% or from 0.02% to 1% by weight of the composition.

In some embodiments, the composition comprises a surface protection agent, wherein the surface protection agent is a copolymer of methyl vinyl ether (MVE) with maleic anhydride or acid. In one embodiment the surface protection agent is a copolymer of MVE with maleic acid. The copolymer of MVE with MA can be referred to as PVM/MA (polyvinyl methyl ether/maleic acid). In one embodiment, the copolymer is a linear copolymer comprising alternating units of MVE and maleic anhydride or acid. In one embodiment the copolymer comprises between a 1:4 to 4:1 ratio of MVE:maleic anhydride or acid. In some embodiments, the copolymer comprises a 1:1 ratio of MVE:maleic anhydride or acid, that is, the MVE content in the copolymer is about 50 mole % and the maleic anhydride or acid content in the copolymer is about 50 mole %. In one embodiment, the copolymer comprises a 1:1 ratio of MVE:maleic acid. In one embodiment, the copolymer is a copolymer of MVE with maleic acid. In some embodiments, the copolymer is the acid form of a copolymer of MVE with maleic anhydride. In some cases, a copolymer is prepared between MVE and maleic anhydride and then is fully or partially hydrolyzed, e.g. following co-polymerization to provide the corresponding acid. In one embodiment the copolymer has a molecular weight in the range 100,000 to 2,000,000 e.g. from 500,000 to 1,500,000, from 800,000 to 1,500,000 or from 100,000 to 1,500,000; as with all polymer molecular weights disclosed herein, the molecular weight referred to is a weight average molecular weight, Mw)

In some embodiments, a copolymer for use in compositions disclosed herein is available commercially under the trade name GANTREZ®, including but not limited to GANTREZ® S-97 HSU solution (Mw 1,500,000), GANTREZ® S-97 BF (Mw 1,200,000), GANTREZ® S-96 (Mw 700,000) and GANTREZ® S-95 (Mw 150,000), all of which are copolymers of MVE with maleic acid. In some embodiments, a copolymer for use in compositions disclosed herein is available commercially under the trade name OraRez®, including but not limited to OraRez® W-100L16 solution (Mw 800,000-1,500,000), a copolymer of MVE with maleic acid. In one embodiment the copolymer is GANTREZ® S-97 which is a copolymer of MVE with maleic acid having an approximate molecular weight of 1,200,000 or 1,500,000. GANTREZ® S-97 may be provided in the form of a solid (powder) or in the form of a liquid, such as an aqueous solution e.g. GANTREZ® S-97 HSU solution. In one embodiment, the copolymer comprises a GANTREZ® polymer or an OreRez® polymer with the following structure and below indicated properties:

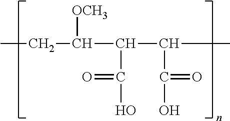

Di-basic acid with pka₁=3.5, pKa₂=6.5

| | Gantrez S-97 | Gantrez S-97 | OraRez |
|---|---|---|---|
| Property | BF | HSU Solution | W-100L16 |
| Appearance @ | White to off- | Slightly | Slightly |
| 25° C. | white, free | hazy viscous | hazy viscous |
| | flowing powder | solution | liquid |
| % Solids | 94 | 15-17 | 15-18 |
| (Active) | | | |
| % Moisture | ≤6 | 85-83 | 85-82 |
| Approx. | 1,200,000 | 1,500,000 | 800,000-1,500,000 |
| Molecular | | | |
| Weight | | | |

The rheological properties of the copolymer can be modified by the addition of salts and bases, as needed. GANTREZ® copolymers are available commercially from Ashland Inc. (Wilmington, DE, USA); ORAREZ® copolymers are available commercially from Boai NKY Pharmaceuticals Ltd. (Jiaozuo, Henan, China), including for example, Orarez W-100 High molecular Weight Polymer, having a molecular weight >1,500,000.

Without being bound by theory, it is a challenge to provide a dentifrice composition that delivers an enhanced fluoridation benefit when the composition comprises a surface protection agent, because the surface protection agent covers sites on the tooth surface where fluoridation typically takes place. As disclosed herein, a surface protection agent, the copolymer of methyl vinyl ether (MVE) with maleic anhydride or acid, can be combined with a source of fluoride ions without adversely impacting delivery of fluoride to the tooth surface, that is the dental enamel. It has now been unexpectedly discovered that controlling the amount of copolymer allows for an improvement with respect to enamel solubility reduction (ESR) without significantly negatively impacting fluoride uptake by the tooth. In one embodiment disclosed herein is a dentifrice composition wherein the copolymer is present from 0.9% to 1.1% by weight of the total composition. In one variation, the copolymer is present in an amount of 0.95% to 1.05% by weight of the total composition. In another variation, the copolymer is present at 1% by weight of the total composition. In another embodiment, the copolymer is used in an amount from 0.05% to 2% by weight of the composition, such as from 0.1% to 1% or from 0.15% to 0.5% or from 0.2% to 0.4% by weight of the composition. In one embodiment the copolymer is used in an amount of about 0.25% by weight of the composition.

As shown herein, it has been found in in vitro testing that when a controlled amount of copolymer is used, a significant improvement may be observed with respect inhibition of demineralization without adversely affecting fluoride uptake. These findings have been supported further by the findings of an in-situ erosion study, also reported herein, where a composition comprising about 0.25% by weight of a methyl vinyl ether maleic acid copolymer, outperformed many other dentifrice compositions tested, with respect to fluoride uptake, remineralization enhancement and demineralization inhibition. As further shown herein using a 5-day erosive acid pH cycling treatment providing improved modeling of dentifrice composition use, a composition comprising sodium lactate and between 0.9% and 1.1% copolymer of MVE with maleic acid by weight of the composition provided more robust surface microhardness reduction than the corresponding composition comprising sodium lactate and 0.25% copolymer by weight of the composition, as well as notable enamel fluoride uptake, following 5-day erosive acid pH cycling treatment. Additionally, as shown herein, a composition comprising sodium lactate and between 0.9% and 1.1% copolymer of MVE with maleic acid by weight of the composition, wherein the copolymer has a weight average molecular weight below 1,500,000 provided even more robust surface microhardness reduction than the corresponding composition comprising sodium lactate and 1% copolymer by weight of the composition, wherein the copolymer has a weight average molecular weight greater than 1,500,000, as well as notable enamel fluoride uptake, following 5-day erosive acid pH cycling treatment. It was thus surprisingly found that using a copolymer having a molecular weight below 1,500,000 yielded better results with regard to surface microhardness reduction and enamel fluoride uptake.

In one embodiment the composition has a slurry pH of about 6.2. In another embodiment, disclosed herein is dentifrice composition comprising a copolymer of MVE with maleic acid. In one embodiment, the copolymer is a 1:1 copolymer of MVE:MA. In another embodiment, the 1:1 the copolymer is present from 0.9% to 1.1% by weight of the total composition or in an amount of 0.95% to 1.05% by weight of the total composition or the copolymer is present at 1% by weight of the total composition.

In one embodiment, a composition disclosed herein does not comprise stannous ions and/or zinc ions. For example, in one embodiment the composition does not comprise from about 0.001% to about 5% of metal ions wherein the metal ions comprise at least 0.001% of stannous ions and optionally from about 0.001% to about 4% of zinc ions. In one embodiment the composition does not comprise a metal compound or salt that becomes more soluble at acidic pH. In one embodiment the composition does not comprise a calcium or zinc compound or salt.

Compositions disclosed herein may contain formulating agents such as dental abrasives, surfactants, thickening agents, humectants, flavoring agents, sweetening agents, opacifying or coloring agents, preservatives and water. It is to be understood that any formulating agent not inconsistent with the technical objectives of the present disclosure may be used in a dentifrice composition disclosed herein. The dentifrice compositions disclosed herein can further contain other oral care actives, including but not limited to a desensitizing agent, a whitening agent, and/or an oral malodor agent.

In some embodiments, the dentifrice composition disclosed herein comprises a dental abrasive; suitable abrasives include but are not limited to silica abrasives, such as those marketed under the trade names Zeodent® (Evonik (Parsippany, NJ, USA)) Sylodent® (Evonik), Sorbosil® (Surfachem Group Ltd, West Yorkshire, UK) or Tixosil® (Solvay, Alorton, IL, USA). The silica abrasive is present in an amount sufficient to ensure adequate cleaning of teeth by the dentifrice composition while not promoting abrasion of teeth. In one embodiment, the silica abrasive is present in an amount up to 15% by weight of the total composition, for example from 2% to 10% by weight; in another embodiment, the silica abrasive is present in at least 5% by weight, for example from 5% to 7% by weight, or 6% by weight of the total composition. Reducing the level of silica abrasive lowers the abrasivity of the dentifrice and minimizes any interaction of the abrasive with fluoride ions, thereby increasing the availability of free fluoride ions.

In some embodiments, the dentifrice composition disclosed herein comprises one or more surfactants. Such surfactants include amphoteric surfactants, for example, long chain alkyl betaines and long chain alkyl amidoalkyl betaines, such as cocamidopropylbetaine; or low ionic surfactants such as sodium methyl cocoyl taurate, which is marketed under the trade name Adinol™ CT by Croda (East Yorkshire, England), or mixtures thereof. In one embodiment, an amphoteric surfactant is used as sole surfactant; in another embodiment, an amphoteric surfactant is combined with a low ionic surfactant. In one embodiment, the surfactant is not a C₁₀₋₁₈ alkyl sulphate surfactant, such as sodium lauryl sulphate, commonly used in oral compositions. In one embodiment, the surfactant is present in the range 0.1% to 10% by weight of the total composition. In another embodiment, the surfactant is present in the range 0.1% to 5% by weight of the total composition. In yet another embodiment, the surfactant is present in the range 0.5% to 1.5% by weight of the total composition.

In some embodiments, the dentifrice composition disclosed herein comprises one or more thickening agents. Such thickening agents include, but are not limited to, nonionic thickening agents such as, for example, (C1-6)alkylcellulose ethers, for instance methylcellulose; hydroxy (C1-6)alkylcellulose ethers, for instance hydroxyethylcellulose and hydroxypropylcellulose; (C2-6)alkylene oxide modified (C1-6)alkylcellulose ethers, for instance hydroxypropyl methylcellulose; and mixtures thereof. Other thickening agents such as natural and synthetic gums or gum like material such as Irish Moss, xanthan gum, gum tragacanth, sodium carboxymethylcellulose, polyvinyl pyrrolidone, starch and thickening silicas may also be used. In one embodiment, the thickening agent is mixture of a thickening silica and xanthan gum. In another embodiment, the thickening agent is present in the range 0.1% to 30% by weight of the total composition. In yet another embodiment, the thickening agent is present in the range 1% to 20% by weight of the total composition. In another embodiment, the thickening agent is present in the range 5% to 15% by weight of the total composition.

In some embodiments, the dentifrice composition disclosed herein comprises one or more humectants including, but not limited to glycerin, xylitol, sorbitol, propylene glycol, polyethylene glycol, or mixtures thereof. In one embodiment, the humectant is present in the range from 10% to 80% by weight of the total composition. In another embodiment, the humectant is present in the range from 20% to 60% by weight of the total composition. In yet another embodiment, the humectant is present in the range from 25% to 50% by weight of the total composition.

In some embodiments, the dentifrice composition disclosed herein comprises one or more opacifying agents, which can be used to enhance the visual appearance of the composition. In some embodiments, the opacifying agent is titanium dioxide. In one embodiment, titanium dioxide is present in the range 0.05% to 2% by weight of the total composition. In another embodiment, titanium dioxide is present in the range 0.075% to 0.2% by weight of the total composition. In yet another embodiment, titanium dioxide is present at 0.1% by weight of the total composition.

In some embodiments, the dentifrice composition disclosed herein comprises one or more flavoring agents, including but not limited to essential oils, flavoring aldehydes, esters, alcohols, and similar materials, as well as menthol, carvone and anethole as well as mixtures thereof. Examples of essential oils include spearmint, peppermint, wintergreen, sassafras, clove, sage, eucalyptus, marjoram, cinnamon, lemon, lime, grapefruit and orange. In one embodiment, the flavoring agent is present in an amount ranging from 0.01% to 4% by weight of the composition. In another embodiment, the flavoring agent is present in an amount ranging from 0.1% to 3% by weight of the composition. In yet another embodiment, the flavoring agent is present in an amount ranging from 0.5% to 2% by weight of the composition.

In some embodiments, the dentifrice composition disclosed herein comprises one or more sweetening agents, including but not limited to, sucrose, glucose, saccharin, sucralose, dextrose, levulose, lactose, mannitol, sorbitol, fructose, maltose, xylitol, saccharin salts (e.g. sodium saccharin) acesulfame and mixtures thereof. In one embodiment, sodium saccharin is used as the sweetening agent. In one embodiment, the sweetening agent is present in an amount ranging from 0.005% to 10% by weight of the composition. In another embodiment, the sweetening agent is present in an amount ranging from 0.01% to 3% by weight of the composition. In yet another embodiment, the sweetening agent is present in an amount ranging from 0.1% to 1% by weight of the composition.

In some embodiments, the dentifrice compositions disclosed herein are aqueous dentifrice compositions. Water may make up the balance of the dentifrice composition. In one embodiment, the composition comprises 5% to 80% such as 10% to 60%, 15% to 40% or 20% to 30% by weight water. This amount of water includes the free water which is added plus that amount which is introduced with other components of the dentifrice composition, such as with sorbitol.

In some embodiments, dentifrice compositions disclosed herein are formulated in the form of toothpastes or gels.

In some embodiments, dentifrice compositions disclosed herein comprise one or more desensitizing agents, which, without being bound by theory can help combat dentine hypersensitivity. In some embodiments, the desensitizing agent is a tubule blocking agent, a nerve desensitizing agent or mixtures thereof. Some examples of desensitizing agents are described in WO 02/15809.

In one embodiment, a tubule blocking agent is a strontium salt such as strontium chloride, strontium acetate or strontium nitrate. In some embodiments, the strontium salt is present in an amount from 5% to 15% by weight of the composition. In one embodiment, the tubule blocking agent is an arginine calcium carbonate salt. In some embodiments, the arginine salt is present in an amount ranging from 0.5% to 30% by weight of the composition, such as from 1% to 10% by weight of the composition or from 1% to 10% by weight of the composition. In one embodiment, the arginine calcium carbonate salt is present from 2% to 8% by weight of the composition.

In one embodiment, the tubule blocking agent is a bioactive glass. In one embodiment, the bioactive glass consists of 45% by weight silicon dioxide, 24.5% by weight sodium oxide, 6% by weight phosphorus oxide, and 24.5% by weight calcium oxide. One such bioactive glass is available commercially under the trade name, NOVAMIN, also known as 45S5 BIOGLASS. In one embodiment, the bioactive glass is present in an amount from 1% to 10% by weight of the composition.

In one embodiment, the tubule blocking agent is stannous fluoride. Without being bound by theory, stannous fluoride, through hydrolysis and oxidation reactions, forms insoluble metal salts that precipitate in dentinal tubules and on the dentine surface to provide effective relief from dentine hypersensitivity. In some embodiments, stannous fluoride is also the source of fluoride, which without being bound by theory, delivers protection from caries and plaque/gingivitis.

In some embodiments, the nerve desensitizing agent is a potassium salt, such as potassium citrate, potassium chloride, potassium bicarbonate, and potassium gluconate. In one embodiment, the nerve desensitizing agent is potassium nitrate. A desensitizing amount of a potassium salt is generally between 2 to 8% by weight of the total composition, for example 5% by weight of potassium nitrate can be used.

In some embodiments, dentifrice compositions disclosed herein comprise a whitening agent. It is to be understood that any whitening agent not inconsistent with the technical objectives of the present disclosure may be used in a dentifrice composition disclosed herein. In one embodiment a whitening agent is a polyphosphate, e.g. sodium tripolyphosphate (STP). a In another embodiment, a silica abrasive, such as those having high cleaning properties serves as a whitening agent. In one embodiment, STP is present in an amount from 2% to 15% or from 5% to 10% by weight of the total composition.

In some embodiments, compositions disclosed herein further comprise an oral malodor agent, including, but not limited to a zinc salt, such as zinc oxide or chloride.

In one embodiment, a composition disclosed herein is suitable for containing in and dispensing from an aluminum-plastic laminate tube or a plastic pump.

In some embodiments, compositions disclosed herein are prepared by admixing the ingredients in the appropriate relative amounts as disclosed herein in any order that is convenient and then adjusting the pH as taught hereinto give a desired pH.

In one aspect, disclosed herein is an exemplary dentifrice composition comprising: an alkali metal salt of lactic acid such as sodium lactate in an amount from 0.5% to 5.0%; a source of free fluoride ions such as sodium fluoride in an amount from 0.05% to 0.5%; a copolymer of MVE with maleic anhydride or acid, such as GANTREZ® S-97, in an amount from 0.05% to 2%; and wherein the composition has a slurry pH in the range greater than 5.0 to less than 6.5.

In another aspect, disclosed herein is a dentifrice composition comprising: a copolymer of MVE with maleic acid; lactic acid or alkali metal salt thereof; and a source of fluoride ions; wherein the copolymer is present in an amount of range 0.9% to 1.1% by weight of the total composition; wherein the lactic acid or alkali metal salt thereof is present in an amount of 1% to 4% by weight of the total composition; and wherein the composition has a slurry pH in the range from 5.0 to 6.5.

In one embodiment, the copolymer is present in an amount of 0.95% to 1.05% by weight of the total composition. In one embodiment, the lactic acid or alkali metal salt thereof is present in an amount of 2% to 3% by weight of the composition. In one embodiment, the alkali metal salt is sodium lactate. In another embodiment, the alkali metal salt, such as sodium lactate, is present in an amount of 2.5% by weight of the total composition.

In one embodiment, the source of free fluoride ions is an alkali metal fluoride. In one embodiment, the alkali metal fluoride is sodium fluoride. In one embodiment, the alkali metal fluoride, optionally sodium fluoride, is present in an amount of 0.05% to 0.5% by weight of the composition. In another embodiment the alkali metal fluoride, optionally sodium fluoride, is present in an amount of 0.2% to 0.3% by weight of the composition.

In one embodiment, the composition has a slurry pH in the range from 5.4 to 6.3. In another embodiment, the composition has a slurry pH in the range from 6.1 to 6.3. In another embodiment, the composition comprises a pH modifying agent, such as for example, sodium hydroxide.

In another embodiment, the copolymer of MVE with maleic acid is a 1:1 copolymer. In another embodiment, the copolymer is present at 1% by weight of the total composition. In yet another embodiment, the copolymer has a molecular weight in the range 100,000 to 2,000,000.

In one embodiment, the dentifrice composition further comprises a desensitizing agent, such as, for example, potassium nitrate. In one embodiment, the desensitizing agent is present in an amount of 2 to 8% by weight of the total composition, or 5% by weight of the total composition.

In one embodiment, the composition further comprises a surfactant, such as, for example cocamidopropylbetaine. In one embodiment, the surfactant, optionally cocamidopropylbetaine, is present in an amount of 0.1% to 5%, such as or in the range 0.5% to 1.5%, or 0.95% to 1.05% by weight of the total composition.

In one embodiment, the surface microhardness reduction of enamel treated with the composition following a 5 day pH cycling treatment with 1% citric acid solution (w/w) at a pH of 3.9 is less than 55%, 54%, 53%, 52%, 51%, 50%, 49% or 48%, that is the percent change in surface microhardness measured after the cycling treatment is between −48% and −55%. In some embodiments, the surface microhardness reduction is less than 50%, that is the percent change following cycling treatment is between 0 and −50%. In another embodiment, the Sound Enamel Hardness retention of enamel treated with the composition following a 5 day pH cycling treatment with 1% citric acid solution (w/w) at a pH of 3.9 is at least 45%, 46%, 47%, 48%, 49%, 50%, 51% or 52%. In yet another embodiment, the enamel fluoride concentration of enamel treated with the composition following 5 days of pH cycling treatment with 1% citric acid solution at a pH of 3.9 is at least 170, 171, 172, 173, 174, 175, 176, 177, 178, 179, 180, 181, 182, 183, 184, or 185 ppm.

In another aspect, any composition hereinbefore defined is used in protecting teeth against dental erosion. In yet another aspect, any composition as hereinbefore defined is used in protecting teeth against dental caries.

In yet another aspect, any composition as hereinbefore defined is used in the treatment and/or inhibition of dental erosion on a dental surface. In yet another aspect, any composition as hereinbefore defined is used in the treatment and/or inhibition of caries on a dental surface.

In one aspect, disclosed herein is a method for protecting teeth against dental erosion which comprises applying an effective amount of a composition disclosed herein to an individual in need thereof. In another aspect, disclosed herein is a method for protecting teeth against dental caries which comprises applying an effective amount of a composition disclosed hereinto an individual in need thereof.

In yet another aspect, disclosed herein is a method for the treatment and/or inhibition of dental erosion on a dental surface, comprising contacting the dental surface with a composition disclosed herein.

In yet another aspect, disclosed herein is a method for the treatment and/or inhibition of dental caries on a dental surface, comprising contacting the dental surface with a composition disclosed herein.

Some embodiments are further illustrated in the following non-limiting Examples.

### EXAMPLES

### Example 1

A dentifrice composition (Formulation 1) as described in Table 1 was prepared as follows. To a suitable vessel was added purified water, sorbitol and glycerin. Then sodium hydroxide, sodium lactate solution, sodium saccharin, sodium fluoride, potassium nitrate, Gantrez, titanium dioxide, and 20% of the flavor were added and mixed with high shear until solids were dissolved. Whilst mixing under vacuum the dental silica was added, then mixed until wetted out. The cocamidopropyl betaine solution and the remaining 80% of the flavor were added and mixed. Separately in a pre-mix vessel, the xanthan gum was mixed with approximately 95% of the polyethylene glycol to form a slurry. Under vacuum this slurry was added to the main vessel whilst mixing under high shear. The remainder of the polyethylene glycol was added into the pre-mix vessel and the resulting mixture was flushed into the main vessel. The resulting paste was mixed under vacuum until homogenous then transferred to suitable tubes.

TABLE 1
Formulation 1

| Ingredient Name | % w/w |
|---|---|
| USP Water | 25.7322 |
| Sorbitol (70% w/w) | 30.0000 |
| Silica (thickening + abrasive) | 17.0000 |
| Glycerin | 8.0000 |
| Potassium Nitrate | 5.0000 |
| Sodium Lactate solution (60% w/w) | 4.1466 |
| Polyethylene Glycol | 3.0000 |
| 47% (aq) Cocamidopropyl | 2.0940 |
| Betaine Solution | |
| Gantrez S-97 HSU | 1.5200 |
| solution (16.5% w/w) | |
| Flavour | 1.2000 |
| Titanium Dioxide | 0.9000 |
| Xanthan Gum | 0.8000 |
| Sodium Saccharin | 0.3000 |
| Sodium Fluoride | 0.2542 |
| Sodium Hydroxide | 0.0530 |
| Total | 100.0000 |

pH of Formulation 1 (1:3 slurry in water)=6.2

### Example 2

### Enamel Fluoride Uptake (EFU)

This example describes an enamel fluoride uptake study carried out on dentifrice compositions as defined below.

### Preparation of Dentifrice Compositions

Formulations 2-4 were prepared having compositional details as provided for in Table 2:

TABLE 2
Compositional Details of Test and Control Dentifrices

| | Formulation 2 | Formulation 3* | Formulation 4* |
|---|---|---|---|
| Ingredient | (Control) | (Test) | (Test) |
| Water | 32.1032 | 39.2348 | 39.3348 |
| Sorbitol (70% w/w) | 30.0000 | 36.0000 | 36.5000 |
| Glycerin | 8.0000 | 2.0000 | 2.0000 |
| PEG 300 (PEG-6) | 3.0000 | 0.4500 | 0.4500 |
| Dental Silica | 18.0000 | 16.0000 | 16.0000 |
| Saccharin Sodium | 0.3000 | 0.3000 | 0.3000 |
| Sodium Fluoride | 0.3152 | 0.3152 | 0.3152 |
| Xanthan gum | 0.8000 | 0.8000 | 0.8000 |
| Carrageenan | — | 0.4000 | 0.4000 |
| Flavour | 1.1000 | 1.0000 | 1.0000 |
| Cocamidopropyl | 1.2000 | 0.8000 | 1.2000 |
| Betaine | | | |
| Titanium Dioxide | 0.1000 | 0.7000 | 0.7000 |
| Potassium Nitrate | 5.0000 | — | — |
| Sodium Hydroxide | 0.0816 | — | — |
| Total | 100.0000 | 98.0000 | 99.0000 |

Formulation 2 was a control composition. *Formulations 3 and 4 were initial dentifrice compositions that were used in the subsequent preparation of the slurries. Formulations 3 and 4 varied slightly from Formulation 2 to allow for later addition of acid and slurry pH adjustment. Formulations 3 and 4 (see Table 2 above) recite the % w/w amount of each ingredient present in a “final” dentifrice composition, following subsequent addition to the initial dentifrice composition of carboxylic acid and any pH modifying agent required to provide the desired pH.

### Preparation of Dentifrice Slurries

Dentifrice slurries were prepared using Formulations 2-4. Slurries were prepared consisting of 1 part paste (Formulation 2, 3 or 4) mixed with 3 parts diluent. The diluent was made of 2 parts acid solution and 1 part water. For the “control”, the acid solution was replaced with water. The total quantity of slurry was 36 g in all cases hence the overall slurry composition consisted of 9 g paste: 18 g acid solution: 9 g water. This approach was taken to allow the creation of slurries from a common base that would have the correct constitution as if the paste had contained all the ingredients. For example, if Formulation 3 had contained 2% malonic acid, and been mixed with water only, the concentration in the final slurry would have been 0.5% (9 g paste+27 g water, a four-fold dilution). The addition of 18 g of 1% malonic acid solution to 9 g of a base paste containing no malonic acid plus 9 g water also gives a concentration in the final slurry of 0.5% (18 g malonic acid plus a total of 18 g paste and water, a two-fold dilution of the malonic acid solution). The resulting slurries were then centrifuged at 10,000 rpm (˜16,000 g) for 10 minutes. The compositional details of the slurries and their respective pH values are provided below in Table 3.

TABLE 3
Composition and pH Details of Dentifrice Slurries

| | | | 1% | 1% | |
|---|---|---|---|---|---|
| | | | Malonic | Citric | |
| | Dentifrice | | Acid | Acid | Slurry |
| Slurry | (9 g) | Water | Soln | Soln | pH |
| 1 | Formulation 2 | 27 g | — | — | 7.2 (un- |
| | | | | | adjusted pH) |
| 2 | Formulation 3 | 9 g | 18 g | — | 7.00 |
| 3 | Formulation 4 | 27 g | — | — | 5.50 |
| 4 | Formulation 3 | 9 g | — | 18 g | 5.50 |
| 5 | Formulation 3 | 9 g | 18 g | — | 5.50 |
| 6 | Formulation 3 | 9 g | 18 g | — | 5.75 |
| 7 | Formulation 3 | 9 g | 18 g | — | 5.25 |

### Method

The EFU test procedure was based on Procedure 40 described in the United States Food and Drug Administration (FDA) testing procedures. In the present case, the incipient lesion was formed using 0.1M lactic acid pH 5.0 containing 0.2% w/v polyacrylic acid (Carbopol 907) that was 50% saturated with hydroxyapatite.

Sound, upper, central, bovine incisors were cleaned of all adhering soft tissue. A core of enamel 3 mm in diameter was prepared from each tooth using a hollow-core diamond drill bit under running water. Specimens were embedded in the end of a plexiglass rod using methyl methacrylate, and polished with 600 grit wet/dry paper and then with micro-fine Gamma Alumina. Twelve specimens per group were used in the study.

Each enamel specimen was etched by immersion into 0.5 ml of 1M perchloric acid (HClO₄) solution for 15 seconds with continuous agitation.

The fluoride content of this solution was determined by using a fluoride electrode to determine the background fluoride content of the enamel specimens.

The specimens were once again ground and polished as described above. An incipient lesion was formed in each enamel specimen by immersion into a 0.1 M lactic acid/0.2% Carbopol 907 solution for 24 hours at 37° C. These specimens were rinsed with water and stored in a humid environment until used.

The pH of certain slurries was adjusted with dropwise addition of 1 M hydrochloric acid or 1M sodium hydroxide to achieve the desired pH specified in Table 3. The specimens were immersed into 25 ml of their assigned slurry supernatant with constant stirring (350 rpm) for 30 minutes. Following treatment, the specimens were rinsed with water. One layer of enamel was removed from each specimen by etching as above. The etch solution was analyzed for fluoride (ion-specific electrode) and calcium. The pre-treatment fluoride (indigenous) level of each specimen was then subtracted from the post-treatment value to determine the change in enamel fluoride due to the test treatment.

### Statistical Analyses

Statistical analyses of the individual means were performed with a one-way analysis of variance model. Significance of differences was analyzed by the Student Newman-Keuls test.

### Results

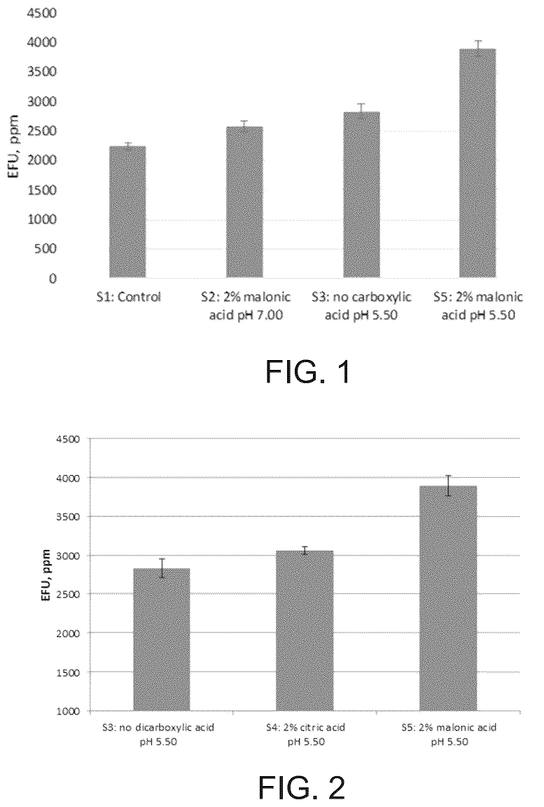

The results of the study are presented in Table 4 (mean EFU±standard error of the mean) and FIGS. 1-3 below.

TABLE 4
Results of EFU Study

| Slurry | Treatment | EFU | s.e. |
|---|---|---|---|
| 1 | Formulation 2 (Control) | 2236 | 58 |
| 2 | Formulation 3 pH 7.00 | 2575 | 99 |
| | 2% malonic acid (Control) | | |
| 3 | Formulation 4 pH 5.50 | 2826 | 118 |
| | no carboxylic acid (Control) | | |
| 4 | Formulation 3 pH 5.50 | 3062 | 51 |
| | 2% citric acid (Control ) | | |
| 5 | Formulation 3 pH 5.50 | 3895 | 133 |
| | 2% malonic acid (Test slurry) | | |
| 6 | Formulation 3 pH 5.75 | 3719 | 129 |
| | 2% malonic acid (Test slurry) | | |
| 7 | Formulation 3 pH 5.25 | 3919 | 127 |
| | 2% malonic acid (Test slurry) | | |

In FIG. 1, at the 5% significance level, all treatments were statistically significantly different to each other. A modest benefit was observed for including the malonic acid at neutral pH, and a slightly greater benefit was observed by reducing the pH to pH 5.5 without adding carboxylic acid by dropwise addition of 1M HCl. By combining the two—pH 5.5 plus carboxylic acid—a substantially greater benefit was observed than either alone demonstrating an unexpected synergy of reducing pH and adding particular carboxylic acid.

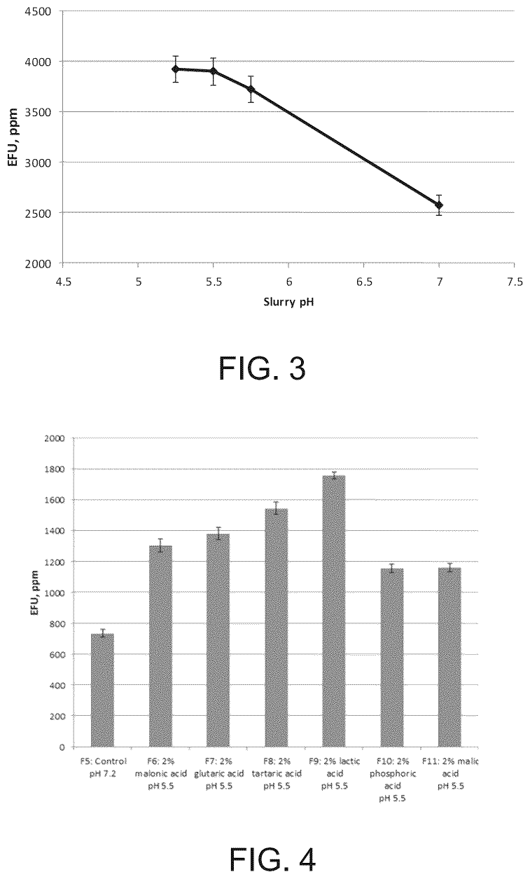

In FIG. 2, the effect of malonic acid at 2% at pH 5.5 was much greater than the effect of citric acid at 2% pH 5.5, demonstrating an unexpected dependence on the nature of the acid used. In FIG. 3, as the pH was decreased the EFU rose until pH 5.5 was reached. There was no further increase in EFU by reducing the pH 5.5 to pH 5.25.

### Conclusion

Synergistic benefit on EFU was observed by reducing the pH to pH 5.5 and adding the carboxylic acid, malonic acid, at 2%. The maximum benefit to EFU in a 2% carboxylic acid was observed at pH 5.5 for malonic acid: below this value EFU did not increase. The boost to EFU from inclusion of malonic acid in these conditions was much greater than the boost from including citric acid.

### Example 3

### Enamel Fluoride Uptake (EFU)

This example describes an enamel fluoride uptake study carried out on dentifrice compositions defined below.

Dentifrice compositions (Formulations 5-11) were prepared (see Table 5 below) and EFU determined as described in Example 2 above. The results are shown in Table 6 and FIG. 4.

TABLE 5
Compositional Details of Test and Control Dentifrices

| | Formulation | | | | | | |
|---|---|---|---|---|---|---|---|
| | 5 | | | | | 10 | 11 |
| | (Control) | 6 | 7 | 8 | 9 | (Control) | (Control) |
| Ingredients: | % w/w | % w/w | % w/w | % w/w | % w/w | % w/w | % w/w |
| Water | 32.1034 | 37.465 | 37.685 | 39.335 | 39.335 | 39.335 | 37.795 |
| Sorbitol | 30.0000 | 36.000 | 36.000 | 35.500 | 35.500 | 35.500 | 36.000 |
| (70% w/w) | | | | | | | |
| Glycerin | 8.0000 | 2.000 | 2.000 | 2.000 | 2.000 | 2.000 | 2.000 |
| PEG 300 | 3.0000 | 0.450 | 0.450 | 0.450 | 0.450 | 0.450 | 0.450 |
| (PEG-6) | | | | | | | |
| Dental | 18.0000 | 16.000 | 16.000 | 16.000 | 16.000 | 16.000 | 16.000 |
| Silica | | | | | | | |
| Saccharin, | 0.3000 | 0.300 | 0.300 | 0.300 | 0.300 | 0.300 | 0.300 |
| sodium | | | | | | | |
| Sodium | 0.3150 | 0.315 | 0.315 | 0.315 | 0.315 | 0.315 | 0.315 |
| fluoride | | | | | | | |
| Xanthan gum | 0.8000 | 0.800 | 0.800 | 0.800 | 0.800 | 0.800 | 0.800 |
| Carrageenan | — | 0.400 | 0.400 | 0.400 | 0.400 | 0.400 | 0.400 |
| Flavor | 1.1000 | 1.000 | 1.000 | 1.000 | 1.000 | 1.000 | 1.000 |
| Cocamido- | 1.2000 | 1.200 | 1.200 | 1.200 | 1.200 | 1.200 | 1.200 |
| propyl | | | | | | | |
| Betaine | | | | | | | |
| Titanium | 0.1000 | 0.700 | 0.700 | 0.700 | 0.700 | 0.700 | 0.700 |
| Dioxide | | | | | | | |
| Potassium | 5.0000 | — | — | — | — | — | — |
| Nitrate | | | | | | | |
| Malonic | — | 2.000 | — | — | — | — | — |
| acid, solid | | | | | | | |
| Glutaric acid | — | — | 2.000 | — | — | — | — |
| Malic acid | — | — | — | — | — | — | 2.000 |
| Tartaric Acid | — | — | — | 2.000 | — | — | — |
| Lactic Acid | — | — | — | — | 2.000 | — | — |
| Potassium | — | — | — | — | — | 2.000 | — |
| dihydrogen | | | | | | | |
| phosphate | | | | | | | |
| Sodium | 0.0816 | 1.370 | 1.150 | — | — | — | 1.040 |
| hydroxide, | | | | | | | |
| solid | | | | | | | |
| Total | 100.0000 | 100.000 | 100.000 | 100.000 | 100.000 | 100.000 | 100.000 |

### Results

TABLE 6
Results of EFU Study

| # | Treatment | EFU | s.e. |
|---|---|---|---|
| 1 | Formulation 5 (Control) | 733 | 23.6 |
| | (pH 7.2) | | |
| 2 | Formulation 6 2% malonic | 1305 | 42 |
| | acid pH 5.5 | | |
| 3 | Formulation 7 2% glutaric | 1379 | 40.2 |
| | acid pH 5.5 | | |
| 4 | Formulation 8 2% tartaric | 1544 | 41.5 |
| | acid pH 5.5 | | |
| 5 | Formulation 9 2% lactic | 1754 | 22.1 |
| | acid pH 5.5 | | |
| 6 | Formulation 10 2% phosphoric | 1154 | 26.4 |
| | acid* pH 5.5 (Control) | | |
| 7 | Formulation 11 2% malic | 1157 | 27 |
| | acid pH 5.5 (Control) | | |

*added as potassium dihydrogen phosphate

At the 5% significance level, all treatments with added acid at pH 5.5 had EFU values statistically significantly greater than the acid-free toothpaste at pH 7.2. The 2% lactic acid product was superior to all other treatments, followed by the 2% tartaric acid product.

The EFU values for the phosphoric acid example and the malic acid example were significantly lower than those observed with the carboxylic acids in Table 6.

### Conclusion

When added to a toothpaste at 2% w/w at pH 5.5, different acids exerted substantially different effect on EFU. Lactic acid was the most effective of those tested. Without being bound by theory the results according to this study demonstrate that a significant effect with respect to fluoride uptake is not achieved merely by formulating a dentifrice composition at an acidic pH (5.5) nor is it achieved merely by using any carboxylic acid; the results observed with phosphoric acid and malic acid were significantly less impressive compared to those observed with the carboxylic acids: malonic, glutaric, tartaric, and lactic acids.

### Example 4

### Enamel Fluoride Uptake (EFU)

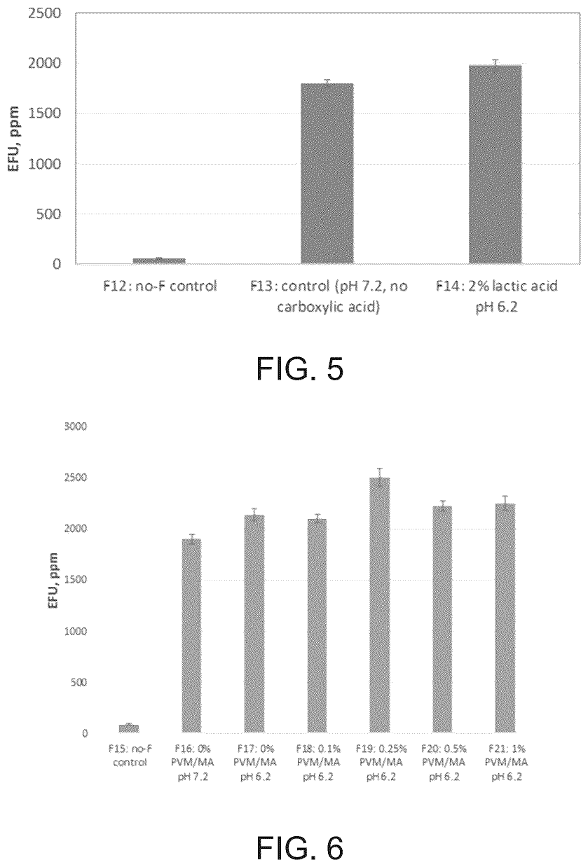

Dentifrice compositions Formulations 12-14 described below (See Table 7) were prepared and EFU determined as described in Example 2 above. The results are shown in Table 8 and FIG. 5.

TABLE 7
Formulations 12-14

| | Formulation | | |
|---|---|---|---|
| | 12 (Control) | 13 (Control) | 14 |
| Ingredient | % w/w | % w/w | % w/w |
| Water | 25.5194 | 30.4518 | 25.2652 |
| Sorbitol (70% w/w) | 30.0000 | 30.0000 | 30.0000 |
| Dental Silica | 17.0000 | 18.0000 | 17.0000 |
| Glycerin | 8.0000 | 8.0000 | 8.0000 |
| Potassium Nitrate | 5.0000 | 5.0000 | 5.0000 |
| PEG 300 (PEG-6) | 3.0000 | 3.0000 | 3.0000 |
| Sodium Lactate | 4.1466 | — | 4.1466 |
| solution (60% w/w) | | | |
| PVM/MA* Copolymer | 1.5200 | — | 1.5200 |
| 16.5% Solution | | | |
| Saccharin Sodium | 0.3000 | 0.3000 | 0.3000 |
| Sodium Fluoride | — | 0.2542 | 0.2542 |
| Xanthan gum | 0.8000 | 0.8000 | 0.8000 |
| Flavour | 1.2000 | 1.2000 | 1.2000 |
| 47% w/w Cocamidopropyl | 2.0940 | 2.0940 | 2.0940 |
| Betaine solution | | | |
| Titanium Dioxide | 0.9000 | 0.1000 | 0.9000 |
| 10.2% Sodium | 0.5200 | 0.8000 | 0.5200 |
| Hydroxide solution | | | |

*PVM/MA = polyvinyl methyl ether/maleic acid

### Results

TABLE 8
Results of EFU Study

| Formulation | EFU | s.e. |
|---|---|---|
| Formulation 12 (Control - no fluoride) | 52 | 6 |
| Formulation 13 (Control - no carboxylic | 1800 | 38 |
| acid salt or copolymer) | | |
| Formulation 14 | 1978 | 59 |

### Conclusion

Formulation 14 was superior to the fluoride control formulation. Both fluoride-containing formulations were superior to the fluoride-free control formulation.

### Example 5

### EFU Study

Dentifrice compositions Formulations 15-21 described below (see Table 9) were prepared and EFU determined as described in Example 2 above. The results are shown in Table 10 and FIG. 6.

TABLE 9
Formulations 15-21

| | Formulation | | | | | | |
|---|---|---|---|---|---|---|---|
| | 15 | 16 | 17 | 18 | 19 | 20 | 21 |
| Ingredients | % w/w | % w/w | % w/w | % w/w | % w/w | % w/w | % w/w |
| Water | 31.5971 | 31.4458 | 31.2522 | 31.29 | 30.03 | 27.92 | 23.69 |
| Sorbitol | 30.0000 | 30.0000 | 30.0000 | 30.00 | 30.00 | 30.00 | 30.00 |
| (70% w/w) | | | | | | | |
| Dental | 18.0000 | 18.0000 | 18.0000 | 18.00 | 18.00 | 18.00 | 18.00 |
| Silica | | | | | | | |
| Glycerin | 8.0000 | 8.0000 | 8.0000 | 8.00 | 8.00 | 8.00 | 8.00 |
| Potassium | 5.0000 | 5.0000 | 5.0000 | 5.00 | 5.00 | 5.00 | 5.00 |
| Nitrate | | | | | | | |
| PEG 400 | 3.0000 | 3.0000 | 3.0000 | 3.00 | 3.00 | 3.00 | 3.00 |
| (PEG-8) | | | | | | | |
| Cocamido- | 1.2000 | 1.2000 | 1.2000 | 1.20 | 1.20 | 1.20 | 1.20 |
| propyl | | | | | | | |
| Betaine | | | | | | | |
| Flavour | 1.2000 | 1.1000 | 1.2000 | 1.20 | 1.20 | 1.20 | 1.20 |
| Xanthan gum | 0.8000 | 0.8000 | 0.8000 | 0.80 | 0.80 | 0.80 | 0.80 |
| Saccharin, | 0.3000 | 0.3000 | 0.3000 | 0.30 | 0.30 | 0.30 | 0.30 |
| sodium | | | | | | | |
| Sodium | — | 0.2542 | 0.2542 | 0.25 | 0.25 | 0.25 | 0.25 |
| fluoride | | | | | | | |
| Titanium | 0.1000 | 0.1000 | 0.1000 | 0.10 | 0.10 | 0.10 | 0.10 |
| Dioxide | | | | | | | |
| PVM/MA* | — | — | — | 0.61 | 1.52 | 3.03 | 6.06 |
| Copolymer 16.5% | | | | | | | |
| solution | | | | | | | |
| (Gantrez S-97) | | | | | | | |
| 10.2% NaOH | 0.8039 | 0.8000 | — | 0.25 | 0.60 | 1.20 | 2.40 |
| solution | | | | | | | |
| Total | 100.0000 | 100.0000 | 100.0000 | 100.00 | 100.00 | 100.00 | 100.00 |

*PVM/MA = polyvinyl methyl ether/maleic acid

### Results

TABLE 10
EFU values of dentifrice compositions
comprising PVM/MA Copolymer

| Formulation | EFU | s.e. |
|---|---|---|
| Formulation 15 | 86 | 7 |
| Formulation 16 (pH 7.2) | 1896 | 50 |
| Formulation 17 (adjusted to pH 6.2) | 2136 | 62 |
| Formulation 18 (adjusted to pH 6.2) | 2096 | 45 |
| Formulation 19 (adjusted to pH 6.2) | 2498 | 87 |
| Formulation 20 (adjusted to pH 6.2) | 2219 | 55 |
| Formulation 21 (adjusted to pH 6.2) | 2249 | 73 |

At 5% significance level, all fluoride-containing formulations had EFU values statistically significantly greater than the fluoride-free placebo. The formulation containing 0.25% PVM/MA copolymer (Formulation 19) was statistically significantly superior to all other formulations tested. There were no significant differences between the other formulations.

### Conclusion

All fluoride-containing formulations were superior to the fluoride-free placebo. However there was evidence to suggest that use of 0.25% polymer was surprisingly favorable to EFU.

### Example 6

### Enamel Solubility Reduction Study

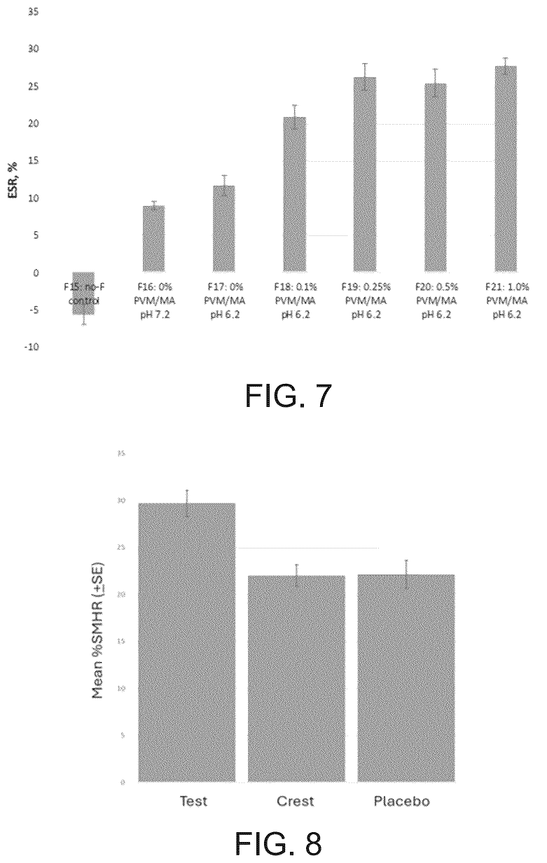

Dentifrice compositions Formulations 15-21 described above in Table 10 were prepared and ESR determined as described below. The results are shown in Table 11 and FIG. 7.

### Tooth Preparation

Three sound human molars were placed in wax so that only the enamel surfaces were exposed, then cleaned and polished. Twelve sets of three teeth each were prepared for the study.

### Lactate Buffer Preparation

A 0.1 M lactic acid solution buffered to pH 4.5 was prepared.

### Deprotection

Teeth surfaces were etched in 0.1 M lactate buffer solution for two one-hour periods at room temperature, then rinsed well with water.

### Pre-Treatment Etch

The test was performed using preheated (37° C.) tooth sets and lactate buffer in an incubator. The acid-pre-treated teeth sets were mounted on the ends of acrylic rods with molten wax. A small hole was drilled in each container lid to accommodate the plastic rod to which the tooth sets were mounted. A 40 ml portion of 0.1 M lactic acid buffer was placed in each container. The rod of the first tooth set will be pushed through the hole in the lid, placed in the first container and adjusted so that all enamel surfaces were immersed into the lactic acid solution. After 15 minutes of stirred exposure to the buffered lactate solution, the tooth sets were removed from the container and rinsed in water. The lactate buffer solutions were retained and analyzed for phosphorus. The tooth sets were then placed back in the 37° C. water bath in preparation for the treatment step.

### Treatment

All tooth sets were treated at the same time (one for each product). The treatment procedure was similar to the etching procedure with the exception of the dentifrice slurry in place of the acid. A 30 ml portion of preheated dentifrice slurry was added to each container, then the teeth were immersed in the dentifrice slurry and stirred for 5 minutes. The other tooth sets were treated in the same manner with the other dentifrice slurries. At the end of treatment, the tooth sets were removed and rinsed well with water.

### Post-Treatment

A second lactic acid exposure was performed by the same method as the pre-treatment etch on the dentifrice-treated samples and the treatment solutions analyzed for phosphorus. The pre- and post-treatment solutions were analyzed for phosphorus using a Klett-Summerson Photelectric Colorimeter.

The tooth sets were the etched again and the procedure repeated additional times so that each tooth set was treated with each dentifrice. The treatments were allocated in a Latin Square design to ensure treatment sequences varied.

### Calculation of E.S.R.

The percent of enamel solubility reduction (ESR) was calculated as the difference between the amount of phosphorus in the pre- and post-acidic solutions, divided by the amount of phosphorus in the pre-solution, multiplied by 100.

### Results

TABLE 11
Results of ESR Study

| # | Sample ID | ESR | s.e. |
|---|---|---|---|
| 1 | Formulation 15 | −5.68 | 1.41 |
| 2 | Formulation 16 | 8.94 | 0.53 |
| 3 | Formulation 17 (adjusted to pH 6.2) | 11.67 | 1.37 |
| 4 | Formulation 18 (adjusted to pH 6.2). | 20.86 | 1.59 |
| 5 | Formulation 19 (adjusted to pH 6.2) | 26.23 | 1.77 |
| 6 | Formulation 20 (adjusted to pH 6.2) | 25.43 | 1.86 |
| 7 | Formulation 21 (adjusted to pH 6.2) | 27.68 | 1.15 |

All fluoride-containing dentifrices gave ESR values statistically superior to the fluoride-free placebo. A clear dose-response to PVM/MA copolymer content was observed between 0% and 0.25%. An approximately 15% increase in ESR was observed due to the presence of 0.25% PVM/MA copolymer. Above 0.25%, no further increase in ESR was observed up to at least 1% PVM/MA copolymer.

### Conclusion

Addition of PVM/MA copolymer up to 0.25% caused a significant increase in enamel solubility reduction. No further increase was noted on adding higher levels of the copolymer.

### Example 7

### Introduction

In order to evaluate the effectiveness of the test formulation a clinical in situ study was performed to compare the effectiveness of the test formulation against a fluoride-free placebo control and a comparator toothpaste also indicated for enamel erosion. The study design employed here has previously been extensively used to investigate the performance of formulations in remineralizing acid-softened enamel [Creeth, 2018; Zero, 2006; Barlow, 2009; Creeth, 2015],

The protocol for the study was posted on the ClinicalTrials.gov website on the 28 Sep. 2017 (Clinicaltrials.gov (Identifier: NCT03296072)).

### Formulations

The test formulation, Formulation 1, is described in Example 1. The fluoride-free placebo was an identical formula to the test, but with the fluoride replaced with water, and the comparator toothpaste was Crest ProHealth Sensitivity and Enamel Shield.

### Study Details

This study was a single center, controlled, single-blind (to the dental examiner and specimen analysts), randomized, three-treatment, three-period, cross-over in situ design to test the remineralizing performance of dentifrices. Treatment was provided once and assessed 2 and 4 hours after application. A washout phase of 2 days (using a fluoride-free dentifrice) was implemented prior to each treatment visit.

In this study, subjects were fitted with an intra-oral device that was capable of holding 8 enamel specimens palatally in the mouth. The enamel specimens were cut from bovine permanent incisors and serially polished to a mirror finish. The specimens were demineralized in vitro by contacting with grapefruit juice for 25 minutes. Specimens were then mounted in the intra-oral appliances and worn by the subjects for the duration of the test period. The toothpaste treatments were brushed onto the buccal surfaces of the teeth for 25s, then the resulting slurry was swilled around the mouth for 95s, expectorated and rinsed with water. Four enamel specimens were removed from the appliance 2 hours after treatment, with the remaining 4 specimens removed 4 hours after treatment. The enamel was then immersed in grapefruit juice in vitro a second time.

The amount of remineralization that had occurred was determined through measuring the microhardness of the enamel surface using a Knoop micro indenter. Indents were performed on the sound enamel prior to contact with grapefruit juice, prior to insertion in the mouth, after the 2 or 4 hours remineralization period and after the second grapefruit juice challenge. The length of the indents was used to calculate the percentage surface microhardness recovery (% SMHR) and the percentage relative erosion resistance (% RER) according to the following equations:

$$\% SMHR = [ ( E 1 - R ) / ( E 1 - B ) ] * 100 [ from Gelhard , 1979 ]$$

$$\% RER = [ ( E 1 - E 2 ) / ( E 1 - B ) ] * 100 [ from Corpron , 1986 ]$$

where B=indentation length (μm) of sound enamel at baseline; E1=indentation length (μm) after first grapefruit juice challenge; R=indentation length (μm) after in situ remineralization and E2=Indentation length (μm) after the second grapefruit juice challenge.

The amount of fluoride incorporated into the remineralized lesion (Enamel fluoride uptake (EFU)) was also chemically determined (using the method of Sakab [Sakkab 1984]) after the enamel specimens had been removed from the mouth, but prior to the second grapefruit juice challenge.

### Results

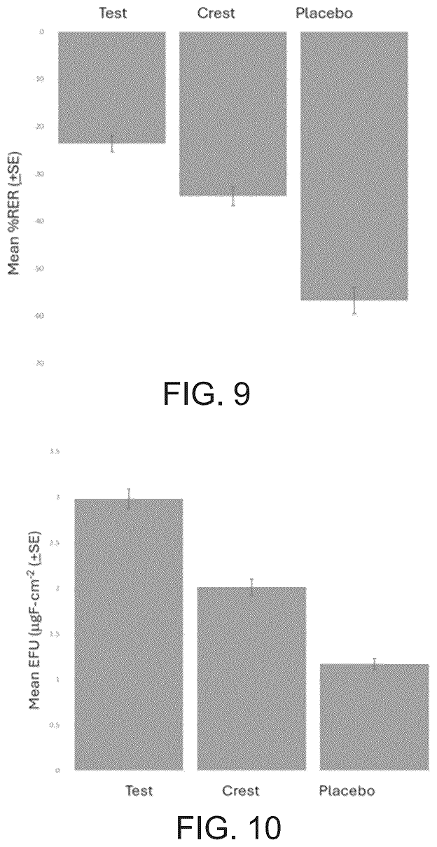

The results are shown in FIGS. 8-10. The test toothpaste showed statistically significantly greater remineralization (as demonstrated by the % SMHR) than either the placebo control or the comparator toothpaste. The test toothpaste also showed statistically superior prevention of demineralization (as shown by % RER) than either the placebo or the comparator toothpaste. In addition, the enamel treated with the test toothpaste had incorporated into the remineralizing lesion (EFU) was statistically superior to the enamel treated with either the fluoride free placebo or the comparator toothpaste.

### Conclusion

The results show that the test toothpaste was more effective at remineralizing acid-softened enamel and at preventing further demineralization than either a fluoride-free control or a comparator product indicated for erosion.

### REFERENCES

Barlow A P, Sufi F, Mason S C. Evaluation of different fluoridated dentifrice formulations using an in-situ erosion remineralization model. The Journal of Clinical Dentistry. 2009; 20(6):192-8.
Corpron R E, Clark J W, Tsai A, More F G, Merrill D F, Kowalski C J, Tice T R, Rowe C E. Intraoral effects of a fluoride-releasing device on acid-softened enamel. The Journal of the American Dental Association. 1986 Sep. 1; 113(3):383-8.
Creeth J E, Kelly S A, Martinez-Mier E A, Hara A T, Bosma M L, Butler A, Lynch R J, Zero D T. Dose-response effect of fluoride dentifrice on remineralisation and further demineralisation of erosive lesions: A randomised in situ clinical study. Journal of Dentistry. 2015 Jul. 1; 43(7):823-31.
Creeth J E, Parkinson C R, Burnett G R, Sanyal S, Lippert F, Zero D T, Hara A T. Effects of a sodium fluoride- and phytate-containing dentifrice on remineralisation of enamel erosive lesions—an in situ randomised clinical study. Clinical oral investigations. 2018 Feb. 8:1-0.
Gelhard T B, Ten Cate J M, Arends J. Rehardening of artificial enamel lesions in vivo. Caries Research. 1979; 13(2):80-3.
Sakkab N Y, Cilley W A, Haberman J P. Fluoride in deciduous teeth from an anti-caries clinical study. Journal of Dental Research. 1984 October; 63(10):1201-5.
Zero D T, Hara A T, Kelly S A, Gonzalez-Cabezas C, Eckert G J, Barlow A P, Mason SC. Evaluation of a desensitizing test dentifrice using an in-situ erosion remineralization model. The Journal of Clinical Dentistry. 2006; 17(4):112-6.

### Example 8

### White Light Interferometry Analysis (Enamel Protection)

### Introduction

The aim of this study was to monitor and quantify the effect, in vitro, of treating human enamel with dentifrice formulations on subsequent erosion by a dietary acid.

The technique of White Light Interferometry can provide rapid visualization of surface topography. Determination of roughness parameters can be carried out in non-contact mode, and height resolution on the nanometer scale is obtainable.

### Test Products

| T1 | Composition of Example 1, Formulation 1 |
|---|---|
| C1 | Competitor toothpaste comprising stannous fluoride |
| | and no Gantrez polymer (Comparator formulation) |
| C2 | Competitor toothpaste comprising sodium fluoride |
| | and no Gantrez polymer (Comparator formulation) |
| C3 | Placebo toothpaste for Formulation 1 which is |
| | fluoride-free and Gantrez -free |

C1—Crest Prohealth Smooth Formula Toothpaste (Ingredients: Stannous Fluoride 0.454% (0.14% W/V Fluoride Ion), Water, Sorbitol, Hydrated Silica, Sodium Lauryl Sulfate, Carrageenan, Sodium Gluconate, Flavor, Xanthan Gum, Zinc Citrate, Stannous Chloride, Sodium Hydroxide, Sodium Saccharin, Sucralose, Titanium Dioxide, Blue 1)

C2—Colgate Enamel Health Toothpaste (Ingredients: Potassium nitrate 5%, Sodium fluoride 0.24% (0.15% w/v fluoride ion) water, sorbitol, hydrated silica, glycerin, PEG-12, tetrasodium pyrophosphate, sodium lauryl sulfate, flavor, microcrystalline cellulose, zinc phosphate, cellulose gum, cocamidopropyl betaine, benzyl alcohol, sodium saccharin, xanthan gum, mica, titanium dioxide, FD&C blue no. 1).

### Methodology

Twenty Human Enamel specimens were polished flat and a region of their surface taped off using acid resistant tape. The specimens were then divided into four treatment groups (n=5 for each group) and immersed into one of dentifrice slurries (1:3 wt % in deionized water) with manual brushing for 2 minutes. Samples were then washing for 1 minute with deionized water. After dentifrice treatment, specimens were suspended in 1% citric acid, pH 3.8 for 5 minutes, without agitation. Specimens were washed with deionized water and air dried then analyzed using white light interferometry.

The surface topography of the specimens was investigated using an ADE PhaseShift MicroXAM White Light Interferometer. Data was acquired from multiple areas (of size 687 μm×511 μm and 215 μm×160 μm) for each specimen. After removal of the tape mask, additional measurements were made to assess bulk tissue loss. Statistical analysis was carried out using a two tailed, unequal variance Student T-Test to >95% confidence level.

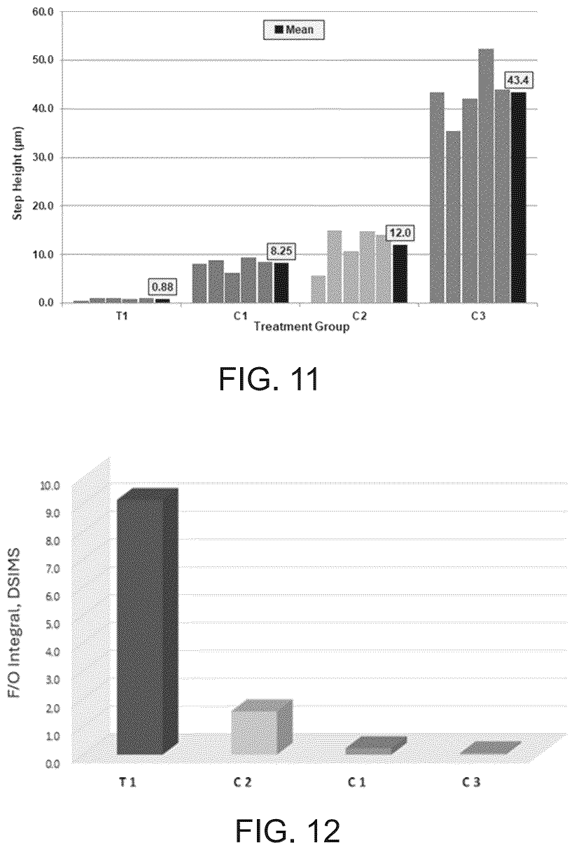

The results are show in in FIG. 11.

The material loss for the treatment groups followed the trend:

$$[ Largest Step ] C 3 > C 2 > C 1 > T 1 [ Smallest Step ] .$$

The step height differences between all treatment groups are statistically significant at a 95% confidence level.

The surface roughness (Sa) for the treatment groups followed the trend:

$$[ Largest Sa ] C 3 > C 2 > C 1 > T 1 [ Smallest Sa ]$$

The Sa differences between all treatment groups are statistically significant at a 95% confidence level except in the case of C2 and C1.

### Conclusion

The above data indicate that pre-treatment with T1 offer the greatest protection against an erosive challenge, followed by pre-treatment with C1, followed by pre-treatment with C2, with the lowest protection offered by pretreatment with C3.

### Example 9

### Dynamic Secondary Ion Mass Spectrometry (Fluoride Uptake)

### Introduction

Dynamic Secondary Ion Mass Spectrometry (DSIMS) can be used to semi-quantitatively determine elemental depth profiles of materials, with nanometer scale resolution. This technique has been used to determine the extent of fluoride and calcium uptake into human enamel surfaces after treatment of erosive lesions with dentifrices and mouthrinses. The aim of this study was to determine the extent of fluoride uptake into human enamel artificial erosive lesions after treatment with the four dentifrices investigated in the white light interferometry study detailed above.

Twenty Human Enamel specimens were polished and suspended in 1% citric acid, pH 3.8 for 5 minutes, without agitation to create artificial erosive lesions. After washing with deionized water, specimens were divided into 4 treatment groups (n=5) and immersed into dentifrice slurries (1:3 wt %) for 2 minutes before washing for 1 minute with deionized water. After treatment, specimens were air dried and analyzed using fluoride DSIMS.

DSIMS imaging analysis was carried out with a Cameca ims 6f instrument using a 15 keV O₂⁺ primary ion beam (˜50 pA) and an electron gun for charge compensation. Images were acquired from areas measuring 100 μm×100 μm. Negative secondary ion detection was used with a nominal extraction field of −5.0 keV. Fluorine/Oxygen integral values were determined for a depth range of 50 μm, i.e. a measure of the relative uptake of fluoride into the upper 50 μm of the tooth enamel surface. A graphical comparison of the results of fluoride uptake across the four treatment groups is shown in FIG. 3.

### Test Products (Same as for Example 8)

| T1 | Composition of Example 1, Formulation 1 |
|---|---|
| C1 | Competitor toothpaste comprising stannous fluoride |
| | and no Gantrez polymer (Comparator formulation) |
| C2 | Competitor toothpaste comprising sodium fluoride |
| | and no Gantrez polymer (Comparator formulation) |
| C3 | Placebo toothpaste for Formulation 1 which is |
| | fluoride-free and Gantrez -free |

### Methodology

Twenty Human Enamel specimens were polished and suspended in 1% citric acid, pH 3.8 for 5 minutes, without agitation to create artificial erosive lesions. After washing with deionized water, specimens were divided into 4 treatment groups (n=5) and immersed into dentifrice slurries (1:3 wt %) for 2 minutes before washing for 1 minute with deionized water. After treatment, specimens were air dried and analyzed using fluoride DSIMS.

DSIMS imaging analysis was carried out with a Cameca ims 6f instrument using a 15 keV O₂⁺ primary ion beam (˜50 pA) and an electron gun for charge compensation. Images were acquired from areas measuring 100 μm×100 μm. Negative secondary ion detection was used with a nominal extraction field of −5.0 keV. Fluorine/Oxygen integral values were determined for a depth range of 50 μm, i.e. a measure of the relative uptake of fluoride into the upper 50 μm of the tooth enamel surface. A graphical comparison of the results of fluoride uptake across the four treatment groups is shown in FIG. 12.

The results of fluoride DSIMS analysis and retrospective line scan analysis showed that fluoride uptake is greatest for specimens treated with the T1 dentifrice, followed by the C2, followed by the C1 dentifrice. Treatment with the C3 dentifrice led to very little fluoride uptake. In order to assess any statistically significant differences in fluoride uptake between the treatment groups, a Student “T” Test was carried out. All differences between the treatment groups were found to be statistically significant.

### Example 10

### Dynamic Secondary Ion Mass Spectrometry (Calcium Uptake)

### Introduction

The aim of this study was to determine the extent of calcium uptake into human enamel artificial erosive lesions after treatment with three dentifrices.

### Test Products (Same as for Example 8)

| T1 | Composition of Example 1, Formulation 1 |
|---|---|
| C1 | Competitor toothpaste comprising stannous fluoride |
| | and no Gantrez polymer (Comparator formulation) |
| C2 | Competitor toothpaste comprising sodium fluoride |
| | and no Gantrez polymer (Comparator formulation) |
| C3 | Placebo toothpaste for Formulation 1 which is |
| | fluoride-free and Gantrez-free |

### Methodology

Twenty Human Enamel specimens were polished and suspended in 1% citric acid, pH 3.8 for 5 minutes, without agitation. After washing with deionized water, specimens were then divided into 4 treatment groups (n=5) and immersed into dentifrice slurries (1:3 wt %) for 2 minutes before washing for 1 minute with deionized water. Enamel specimens from two of the four treatment groups were incubated in the slurry made from the dentifrice T1. The enamel was subsequently placed into an artificial saliva solution for 24 hrs. This solution contained calcium significantly enriched with ⁴⁴calcium (as calcium chloride) for three of the treatments. For enamel in the second dentifrice C3 (placebo for T1) a standard artificial saliva solution was used as a control (identical to the artificial saliva used for the other treatments but containing ⁴⁰calcium as calcium chloride). Specimens were then washed for 1 minute with deionized water, air dried and analyzed using for ⁴⁴calcium DSIMS.

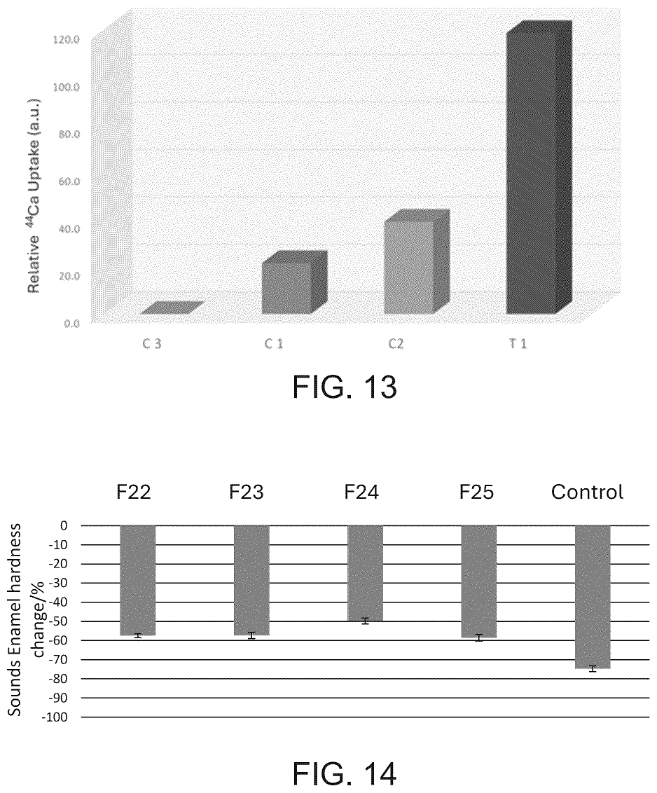

DSIMS imaging analysis was carried out with a Cameca ims 4F instrument utilizing a 15 keV O₂⁺ primary ion beam (˜100 pA). Images for the species ⁴⁰Ca, ⁴²Ca, ⁴⁴Ca and ⁴⁰Ca¹⁹F were acquired from a minimum of two areas per sample measuring, typically, 100 μm×100 μm. Positive secondary ion detection was used with an extraction field at the sample surface of +4.5 keV and a normal incidence electron gun for charge compensation. Linescan data was subsequently obtained from each image using the Cameca ims 4f data processing software. A graphical representation of the results is shown in FIG. 13.

### Results

DSIMS imaging of the enamel and retrospective line scan analysis showed that ⁴⁴calcium uptake was negligible in the specimens treated with C3 but subsequently incubated in the artificial saliva solution composed of calcium of normal isotopic composition. For the specimens incubated in the ⁴⁴calcium-enriched artificial saliva, the extent of ⁴⁴calcium incorporation was greatest for specimens pre-treated with the T1 dentifrice, followed by those treated with the C2 dentifrice, followed by the C1 dentifrice (FIG. 13). Although 44calcium uptake occurs to a depth of greater than 20 μm for the first three treatments, the greatest inter-group differences in mean ⁴⁴calcium uptake occur in the upper ˜10 μm of the enamel surface. In this region treatment with T1 leads to a ⁴⁴calcium uptake approximately three and a half times higher than treatment with C2 and approximately five times higher than treatment with C1. C2 has a ⁴⁴calcium uptake ˜1.5 times higher than treatment with C1. In order to assess any statistically significant differences in ⁴⁴calcium uptake between treatment groups, a Student “T” Test has been carried out. All differences in calcium uptake observed with statistically significant.

### Conclusion

The greater calcium uptake value observed for the test dentifrice (Tl) relative to the comparator formulations (C1 and C2) is indicative of enhanced remineralization of the tooth enamel surface for the test dentifrice.

### Example 11

The objective of this study was to determine the efficacy of dentifrices in preventing the erosive softening of enamel substrate caused by repeated exposure to dietary acids (citric acid) in a pH cycling in vitro study, which more closely correlates with the effect of using dentifrice compositions. Erosive softening effects were measured via surface microhardness assessments, with additional enamel fluoride concentration assays following a 5-day in vitro pH cycling treatment regimen, as described below. Formulations 22-25 described in Table 12 were prepared according to the method of Example 1. The results are shown in Tables 14-15 and FIGS. 14-16.

TABLE 12
Formulations 22-25

| | Formulation | | | |
|---|---|---|---|---|
| | 22 | 23 | 24 | 25 |
| Ingredient Name | % w/w | % w/w | % w/w | % w/w |
| Water, Purified | 26.0326 | 22.4656 | 21.0656 | 21.0756 |
| Sorbitol, Liquid | 30.0000 | 30.0000 | 30.0000 | 30.0000 |
| Glycerol | 8.0000 | 8.0000 | 8.0000 | 8.0000 |
| Polyethylene | 3.0000 | 3.0000 | 3.0000 | 3.0000 |
| Glycol 300 | | | | |
| Potassium Nitrate | 5.0000 | 5.0000 | 5.0000 | 5.0000 |
| Sodium Fluoride | 0.2542 | 0.2542 | 0.2542 | 0.2542 |
| Silica Dental | 13.0000 | 13.0000 | 13.0000 | 13.0000 |
| Type (Medium | | | | |
| Thickening) | | | | |
| Silica, Dental | 4.0000 | 4.0000 | 4.0000 | 4.0000 |
| Type (Low | | | | |
| Abrasion Silica) | | | | |
| Sodium Lactate | 4.1466 | 4.1466 | 4.1466 | 4.1466 |
| Solution 60% | | | | |
| PVM/MA Copolymer | 1.5200 | 4.8500 | 6.2500 | 0.0000 |
| (16.5% Solution) | | | | |
| PVM/MA Copolymer | 0.0000 | 0.0000 | 0.0000 | 6.2500 |
| (16.5% Solution) | | | | |
| High molecular | | | | |
| Weight Polymer | | | | |
| (Mw > 1,500,000) | | | | |
| Sodium Hydroxide | 0.0530 | 0.2900 | 0.2900 | 0.2800 |
| 47% (aq) | 2.0936 | 2.0936 | 2.0936 | 2.0936 |
| Cocamidopropyl | | | | |
| Betaine Solution | | | | |
| Titanium | 0.6000 | 0.6000 | 0.6000 | 0.6000 |
| Dioxide | | | | |
| Xanthan Gum | 0.8000 | 0.8000 | 0.8000 | 0.8000 |
| Saccharin | 0.3000 | 0.3000 | 0.3000 | 0.3000 |
| Sodium | | | | |
| Flavour | 1.2000 | 1.2000 | 1.2000 | 1.2000 |
| Total | 100.000 | 100.000 | 100.000 | 100.000 |

Enamel specimens (˜4 mm in diameter) were removed from extracted human teeth and mounted in acrylic blocks. The specimens were mounted on top as flat as possible. The methacrylate was then allowed to cure. Once cured, the surface of the enamel specimen was serially ground and polished with 600, 800 grit wet Al₂O₃ paper and then further polished with 3 μm and 1 μm monocrystalline diamond suspension. The surface microhardness (SMH) of each enamel specimen was then determined using a Vickers Hardness diamond indenter (LECO LM 247AT) at a load of 50 gF and 15-second dwell time. The average specimen surface microhardness was determined from four (4) indentations on the surface of each specimen. The specimens were then balanced into treatment groups of N=10 based on their Mean (N=4) Vickers Hardness Number (VHN).

### Remineralizing Solution

A 50:50 mixture of pooled human saliva: artificial saliva w/mucin was utilized as the remineralizing solution for all treatment groups. The artificial saliva comprised: Gastric mucin 2.20 g/L; NaCl 0.381 g/L; CaCl₂·2H₂O 0.213 g/L; KH₂PO₄ 0.738 g/L; and KCl 1.114 g/L. The pH of the artificial saliva was adjusted to 7.0 after all ingredients dissolved completely; the prepared saliva was mixed 50:50 (w/w) with pooled human saliva.

### Erosive Challenge Solution

During erosive acid challenge periods, the specimens were immersed in a 1% citric acid solution (w/w) buffered to a pH of 3.9 with 5M NaOH. Fresh solution was used for each challenge period.

### Test Sample Treatment Preparation

During the test sample treatment periods, the specimens were immersed in dentifrice slurries (1:3 w/w) with deionized water (10.0 g dentifrice & 30.0 g diluent). Fresh samples were prepared just prior to each treatment. Following complete homogenization of the dentifrice/water slurries, the pH of the slurries was determined & recorded using a calibrated digital pH-meter with a sensitivity of ±0.1 mV. When necessary, the pH of each slurry was adjusted to a final pH of 6.2 using dilute HCl or NaOH.

### Treatment Regimen

The cyclic treatment regimen consisted of a 20 minute/day erosive acid challenge (4 challenges at 5 minutes per challenge), followed by remineralizing solution soaks and two, 2-minute dentifrice treatment periods. The remaining time, the specimens were immersed in the remineralizing solution. All treatments were performed at 37° C. The citric acid challenges were performed at static conditions, artificial saliva soaks were stirred at 130 rpm and dentifrice treatments at 350 rpm. Between each treatment phase, the specimens were rinsed with running DI water for a period of 5-seconds. The regimen was repeated for a total of 5-days; the daily schedule is summarized in Table 13, using each of Formulation 22 (“F22”), Formulation 23 (“F23”), Formulation 24 (“F24”), Formulation 25 (“F25”) and a control formulation containing no fluoride (“Control”), see Formulation 12 above.

TABLE 13
Acid Erosion Protocol

| Time point | Step in Protocol |
|---|---|
| 8:00-8:02 am | Dentifrice Treatment 1. |
| 8:02-9:02 am | 5 second DI rinse, artificial saliva soak |
| 9:02-9:07 am | 5 second DI rinse, 5 minute citric acid challenge 1 |
| 9:07-10:07 am | 5 second DI rinse, artificial saliva soak |
| 10:07-10:12 am | 5 second DI rinse, 5 minute citric acid challenge 2 |
| 10:12-12:12 pm | 5 second DI rinse, artificial saliva soak |
| 12:12-12:17 pm | 5 second DI rinse, 5 minute citric acid challenge 3 |
| 12:17-1:17 pm | 5 second DI rinse, artificial saliva soak |
| 1:17-1:22 pm | 5 second DI rinse, 5 minute citric acid challenge 4 |
| 1:22-2:22 pm | 5 second DI rinse, artificial saliva soak |
| 2:22-2:24 pm | 5 second DI rinse, Dentifrice Treatment 2 |
| 2:24 pm-8:00 am | 5 second DI rinse, artificial saliva soak |

### Post-Treatment Surface Microhardness (SMH) Analyses

Following completion of the 5-day pH cycling treatment regimen, the surface microhardness (SMH) of each enamel specimen was determined using a Vickers Hardness diamond indenter (LECO LM 247AT) at a load of 50 gF and 15 second dwell time. The average specimen surface microhardness was determined from four (4) indentations on the surface of each specimen, in close proximity to the pre-treatment indentions. The differences in VHN between the pre- and post-treatment assessments were calculated as ΔVHN.

### Post-Treatment Enamel Fluoride Concentration Assays

Following completion of the post-treatment surface microhardness assessments, the enamel specimens were assayed for enamel fluoride concentration. A layer of enamel was removed from each specimen by immersion in 0.5 ml of 1.0 N HClO₄ for 15-seconds with constant agitation. An aliquot of each solution was buffered with Total Ionic Strength Adjustment Buffer II (TISABII) to a pH of 5.2 (0.25 ml sample, 0.5 ml TISABII and 0.25 ml IN NaOH) and the fluoride concentration determined via fluoride ion selective electrode (Orion™ ISE) by comparison to a similarly prepared standard curve. A second aliquot was analyzed for calcium concentration via atomic absorption spectroscopy (Perkin Elmer AAnalyst 200) for use in depth of etched enamel determination (0.05 ml sample diluted to 5.0 ml). The resultant calculations provided the enamel fluoride concentration as ppm F for each enamel specimen.

### Data Analysis

Statistical analyses were performed by One-Way Analysis of Variance (ANOVA) & Student-Newman-Keuls (SNK) multiple comparison models using Sigma Plot Software (14.5). Statistical significance of all analyses was determined at p<0.050 (95% CI).

### GLP Compliance

This study was conducted in compliance with FDA GLP guidelines.

### Results

TABLE 14
Surface Microhardness Reduction (%)

| | Surface Microhardness |
|---|---|
| Formulation | Reduction (%) |
| 22 | −57.5 ± 1.4 |
| 23 | −57.4 ± 1.6 |
| 24 | −49.8 ± 1.5 |
| 25 | −58.6 ± 1.7 |
| Control | −74.8 ± 1.5 |

### Conclusion

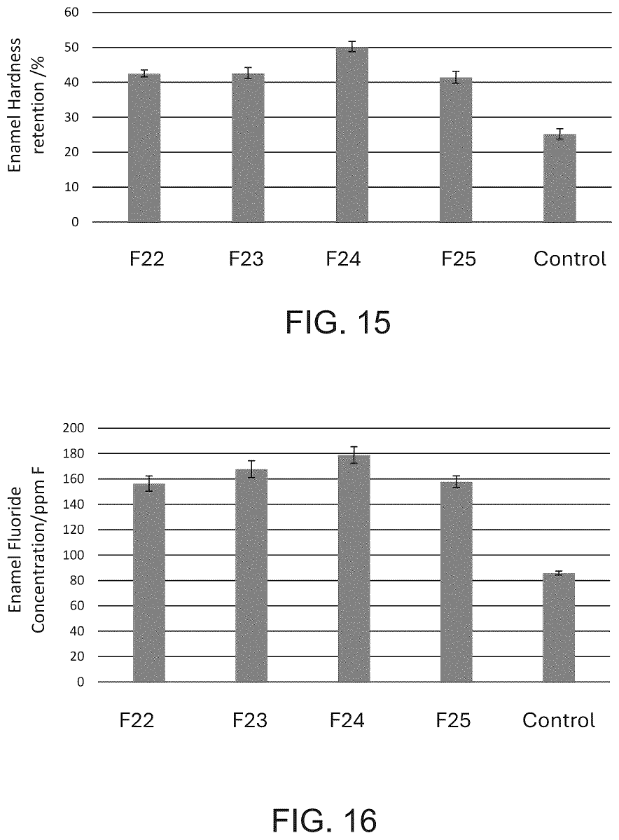

Formulation 24 exhibited a significantly greater efficacy (p≤0.001) in preventing enamel surface softening from the erosive citric acid exposures compared to all other treatment groups (FIG. 14). Enamel treated with Formulation 24 retained a significantly higher hardness after 5 days of cyclic treatment than all other treatment groups, offering the best protection against demineralization (FIG. 15).

The results demonstrate that compositions comprising from 0.9% to 1.1% by weight of poly (methylvinylether/maleic acid) copolymer having a molecular weight ≤1,500,000 and sodium lactate demonstrate significantly greater efficacy in preventing enamel surface softening than comparative compositions. The results further demonstrate that enamel treated with compositions comprising from 0.9% to 1.1% by weight of poly (methylvinylether/maleic acid) copolymer having a molecular weight ≤1,500,000 and sodium lactate retained a significantly higher hardness after five days of cyclic treatment than enamel treated comparatively with comparative compositions. This is indicative of the compositions comprising from 0.9% to 1.1% by weight of poly (methylvinylether/maleic acid) copolymer having a molecular weight ≤1,500,000 and sodium lactate demonstrating better protection against demineralization than comparative compositions comprising lower amounts of protective copolymer or comprising copolymer having a molecular weight greater than 1,500,000.

TABLE 15
Enamel Fluoride concentration

| | Enamel Fluoride |
|---|---|
| Formulation | Concentration (ppm F) |
| 22 | 156.3 ± 6.0 |
| 23 | 167.7 ± 6.7 |
| 24 | 178.8 ± 6.6 |
| 25 | 157.8 ± 4.6 |
| Control | 85.8 ± 1.6 |

### Conclusion

At pH 6.2, Formulation 24 exhibited a greater) enamel fluoride concentration compared to the other treatment groups (FIG. 16).

The results demonstrate that compositions comprising from 0.9% to 1.1% by weight of poly (methylvinylether/maleic acid) copolymer having a molecular weight ≤1,500,000 and sodium lactate imparted a higher enamel fluoride concentration to enamel treated with the compositions than enamel treated comparatively with comparative compositions. This is highly surprising and unexpected in view of the compositions comprising higher amounts of protective copolymer would normally be expected to reduce fluoride uptake into enamel.

The results thus demonstrate that compositions comprising from 0.9% to 1.1% by weight of poly (methylvinylether/maleic acid) copolymer having a molecular weight ≤1,500,000 and sodium lactate can provide both increased enamel protection and also increased fluoride uptake for improved remineralization when compared to compositions comprising lower amounts of protective copolymer or comprising copolymer having a molecular weight greater than 1,500,000.

Additional exemplary embodiments contemplated herein are as follows:

Embodiment 1. A dentifrice composition comprising: a copolymer of MVE with maleic acid; lactic acid or alkali metal salt thereof; and a source of fluoride ions; wherein the copolymer is present in an amount of range 0.9% to 1.1% by weight of the total composition; wherein the lactic acid or alkali metal salt thereof is present in an amount of 1% to 4% by weight of the total composition; and wherein the composition has a slurry pH in the range from 5.0 to 6.5.

Embodiment 2. The composition of Embodiment 1, wherein the lactic acid or alkali metal salt thereof is present in an amount of 2% to 3% by weight of the composition.

Embodiment. 3. The composition of Embodiment 1 or 2, wherein the copolymer is present in an amount of 0.95% to 1.05% by weight of the total composition.

Embodiment 4. The composition of Embodiment 1 or Embodiment 2, wherein the alkali metal salt is sodium lactate.

Embodiment 5. The composition of any of Embodiments 1-4, wherein the alkali metal salt is present in an amount of 2.5% by weight of the total composition.

Embodiment 6. The composition of any of Embodiments 1-5, wherein the source of free fluoride ions is an alkali metal fluoride.

Embodiment 7. The composition of Embodiment 6, wherein the alkali metal fluoride is sodium fluoride.

Embodiment 8. The composition of Embodiment 6 or 7, wherein the alkali metal fluoride is present in an amount of 0.05% to 0.5% by weight of the composition.

Embodiment 9. The composition of any of Embodiments 6-8, wherein the alkali metal fluoride is present in an amount of 0.2% to 0.3% by weight of the composition.

Embodiment 10. The composition of any of Embodiments 1-9, wherein the composition has a slurry pH in the range from 5.4 to 6.3.

Embodiment 11. The composition of any of Embodiments 1-10, wherein the composition has a slurry pH in the range from 6.1 to 6.3.

Embodiment 12. The composition of any of Embodiments 1-11, wherein the composition comprises a pH modifying agent.

Embodiment 13. The composition of Embodiment 12, wherein the pH modifying agent is sodium hydroxide.

Embodiment 14. The composition of any of Embodiments 1-13, wherein the copolymer of MVE with maleic acid is a 1:1 copolymer.

Embodiment 15. The composition of any of Embodiments 1-14, wherein the copolymer is present at 1% by weight of the total composition.

Embodiment 16. The composition of any of Embodiments 1-15, wherein the copolymer has a weight average molecular weight in the range 100,000 to 1,500,000 or in the range 800,000 to 1,500,000.

Embodiment 17. The composition of any of Embodiments 1-16, further comprising a desensitizing agent.

Embodiment 18. The composition of Embodiment 17, wherein the desensitizing agent is potassium nitrate.

Embodiment 19. The composition of Embodiment 17 or 18, wherein the desensitizing agent is present in an amount of 2 to 8% by weight of the total composition.

Embodiment 20. The composition of any of Embodiments 17-19, wherein the desensitizing agent is present in an amount of 5% by weight of the total composition.

Embodiment 21. The composition of any of Embodiments 1-20, further comprising a surfactant.

Embodiment 22. The composition of Embodiment 21, wherein the surfactant is cocamidopropyl betaine.

Embodiment 23. The composition of Embodiment 21 or 22, wherein the surfactant is present in an amount of 0.1% to 5% by weight of the total composition.

Embodiment 24. The composition of any of Embodiments 21-23, wherein the surfactant is present in the range 0.5% to 1.5% by weight of the total composition.

Embodiment 25. The composition of any of Embodiments 21-24, wherein the surfactant is present in the range 0.95% to 1.05% by weight of the total composition.

Embodiment 26. The composition according to any of Embodiments 1-25, wherein the surface microhardness reduction of enamel treated with the composition following a 5 day pH cycling treatment with 1% citric acid solution at a pH of 3.9 is less than less than 55%, 54%, 53%, 52%, 51%, 50%, 49% or 48%.

Embodiment 27. The composition according to any of Embodiments 1-26, wherein the Sound Enamel Hardness retention of enamel treated with the composition following a 5 day pH cycling treatment with 1% citric acid solution at a pH of 3.9 is at least 45%, 46%, 47%, 48%, 49%, 50%, 51% or 52%.

Embodiment 28. The composition according to any of Embodiments 1-27, wherein the enamel fluoride concentration of enamel treated with the composition following 5 days of pH cycling treatment with 1% citric acid solution at a pH of 3.9 is at least 170 ppm, 175 ppm, 180 ppm, or 185 ppm.

Embodiment 29. A method of protecting teeth against dental erosion, comprising administering the composition of any one of Embodiments 1-28.

Embodiment 30. A method of protecting teeth against dental caries, comprising administering the composition of any one of Embodiments 1-28.

Various implementations and embodiments of systems, apparatus, and methods have been described in fulfillment of the various objectives of the present disclosure. It should be recognized that these implementations and embodiments are merely illustrative of the principles of the present disclosure. Numerous modifications and adaptations thereof will be readily apparent to those skilled in the art without departing from the spirit and scope of the present disclosure. For example, individual steps of methods described herein can be carried out in any manner and/or in any order not inconsistent with the objectives of the present disclosure, and various configurations or adaptations of apparatus described herein may be used.

1. A dentifrice composition comprising:
a copolymer of MVE with maleic acid;
lactic acid or alkali metal salt thereof; and
a source of fluoride ions;
wherein the copolymer is present in an amount of 0.9% to 1.1% by weight of the total composition;
wherein the lactic acid or alkali metal salt thereof is present in an amount of 1% to 4% by weight of the total composition; and
wherein the composition has a slurry pH in the range from 5.0 to 6.5.

2. The composition of claim 1, wherein the lactic acid or alkali metal salt thereof is present in an amount of 2% to 3% by weight of the composition.

3. The composition of claim 1, wherein the copolymer is present in an amount of 0.95% to 1.05% by weight of the total composition.

4. The composition according to claim 1, wherein the alkali metal salt is sodium lactate present in an amount of 2.5% by weight of the total composition.

5. The composition according to claim 1, wherein the source of free fluoride ions is an alkali metal fluoride.

6. The composition according to claim 5, wherein the alkali metal fluoride is sodium fluoride present in an amount of 0.05% to 0.5% by weight of the composition.

7. The composition according to claim 1, wherein the composition has a slurry pH in the range from 5.4 to 6.3.

8. The composition according to claim 1, wherein the composition comprises a pH modifying agent.

9. The composition according to claim 8, wherein the pH modifying agent is sodium hydroxide.

10. The composition according to claim 1, wherein the copolymer of MVE with maleic acid is a 1:1 copolymer.

11. The composition according to claim 1, wherein the copolymer has a molecular weight in the range 100,000 to 1,500,000.

12. The composition according to claim 1, further comprising a desensitizing agent.

13. The composition according to claim 1, wherein the surface microhardness reduction of enamel treated with the composition following a 5 day pH cycling treatment with 1% citric acid solution at a pH of 3.9 is less than 55%.

14. The composition according to claim 1, wherein the enamel fluoride concentration of enamel treated with the composition following 5 days of pH cycling treatment with 1% citric acid solution at a pH of 3.9 is at least 175 ppm.

15. A method of protecting teeth against dental erosion and/or dental caries, comprising administering the composition of claim 1.
